(19) United States
(12) Patent Application Publication
(10) Pub. No.: US 2024/0239915 A1
(43) Pub. Date: Jul. 18, 2024

(54) FC VARIANT WITH HIGH THERMAL STABILITY AND ATTENUATED EFFECTOR FUNCTION

(71) Applicant: ZYMEWORKS BC INC., Vancouver (CA)

(72) Inventors: Naoya Shinozaki, Tokyo (JP); Tomohiro Makino, Tokyo (JP); Nobumi Nagaoka, Tokyo (JP)

(21) Appl. No.: 18/592,409

(22) Filed: Feb. 29, 2024

Related U.S. Application Data
(63) Continuation of application No. PCT/CA2022/051331, filed on Sep. 2, 2022.

(30) Foreign Application Priority Data
Sep. 3, 2021 (JP) 2021-144352

(51) Int. Cl. C07K 16/30

(52) U.S. Cl. CPC C07K 16/30; C07K 2317/524; C07K 2317/92

(57) ABSTRACT

A polypeptide comprising one or more variant Fc regions, each of the variant Fc regions comprising two CH2 domains, wherein at least one of the CH2 domains in at least one of the variant Fc regions is a variant CH2 domain, and wherein amino acid residues at positions 234, 235, and 265 (all positions expressed by EU numbering) of the variant CH2 domain are Ala, Ala, and Gly, or Ala, Ala, and Asn, respectively.

### CROSS REFERENCE TO RELATED APPLICATIONS

This application is a continuation of PCT/CA2022/051331, filed Sep. 2, 2022, which claims the benefit of and priority to Japanese Patent Application No. 2021-144352, filed on Sep. 3, 2021, the disclosure of which is incorporated herein by reference in its entirety for all purposes.

### SEQUENCE LISTING

The instant application contains a Sequence Listing which has been submitted electronically and is hereby incorporated by reference in its entirety. Said XML copy, created on Aug. 30, 2022, is named ZYME086WO_67784.xml and is 37,288 bytes in size.

### FIELD

The present disclosure relates to the field of Fc variants and in particular to a polypeptide comprising a variant IgG Fc region, a molecule comprising the polypeptide, and a polypeptide-drug conjugate in which said polypeptide is conjugated to a drug, which are useful as a medicament.

### BACKGROUND

The Fc region of an antibody provides the antibody with an effector function, such as long half-life in blood and antibody-dependent cellular cytotoxicity (ADCC). When the Fc region is derived from the IgG subclass, which is used in most antibody drugs, the effector function depends on binding of the Fc region to a family of receptors called Fcγ receptors (FcγRs). However, binding activity to FcγR has been suggested to be involved in safety risks, such as infusion reactions (see J Immunotoxicol; 5(1):11-5 (2008)), and thus in some circumstances may be an undesired property for an antibody used as a medicament.

A variety of amino acid substitutions on the Fc region of IgG antibodies that reduce the binding activity to human FcγR have previously been reported. See, for example, International Publication Nos. WO 1988/07089; WO 1999/51642; WO 2000/42072; WO 2013/092001; WO 2015/109131 and WO 2020/086776, and Protein Eng Des Sel; 29(10):457-66 (2016), and J Biol Chem; 292(5):1865-75 (2017).

Amino acid substitutions that reduce binding of the Fc region to FcγR may result in a reduced thermal stability of the Fc region (see, for example, International Publication No. WO 2020/086776 and J Biol Chem; 292(5):1865-75 (2017)).

International Publication No. WO 2013/118858 describes amino acid substitutions in the Fc region that improve thermal stability as well as increase or decrease an effector function. WO 2013/118858 shows that a combination of amino acid substitutions, each of which independently improves thermal stability, does not necessarily improve thermal stability in an additive manner.

### SUMMARY

Described herein are Fc variants with high thermal stability and attenuated effector function. One aspect of the disclosure relates to a polypeptide comprising one or more variant Fc regions, each of the variant Fc regions comprising two CH2 domains, wherein at least one CH2 domain in at least one of the variant Fc regions is a variant CH2 domain comprising amino acid substitutions at positions 234, 235 and 265 (all positions expressed by EU numbering), wherein the amino acid residues at positions 234, 235, and 265 of the variant CH2 domain are Ala, Ala, and Gly, respectively, or Ala, Ala, and Asn, respectively, and wherein each of the one or more variant Fc regions is a variant IgG1 Fc region, a variant IgG4 Fc region or a variant IgG1/IgG4 Fc region.

Another aspect of the disclosure relates to a molecule comprising a polypeptide as described herein.

Another aspect of the disclosure relates to a polypeptide-drug conjugate comprising a polypeptide as described herein conjugated to one or more drugs or modifying agents.

Another aspect of the disclosure relates to a nucleic acid comprising a nucleotide sequence encoding a polypeptide as described herein or a portion thereof.

Another aspect of the disclosure relates to a host cell comprising a nucleic acid that comprises a nucleotide sequence encoding a polypeptide as described herein or a portion thereof.

Another aspect relates to a method for producing a polypeptide as described herein comprising culturing a host cell comprising a nucleic acid that comprises a nucleotide sequence encoding the polypeptide or a portion thereof in a medium under conditions suitable for expression of the polypeptide, and recovering the polypeptide from the medium or the host cell.

Another aspect of the disclosure relates to a pharmaceutical composition comprising a polypeptide as described herein, a molecule comprising the polypeptide, or a polypeptide-drug conjugate comprising the polypeptide conjugated to a drug or a modifying agent, and a pharmaceutically acceptable carrier.

Another aspect relates to a use of a polypeptide as described herein, a molecule comprising the polypeptide, or a polypeptide-drug conjugate comprising the polypeptide conjugated to a drug or a modifying agent, in therapy.

### BRIEF DESCRIPTION OF DRAWINGS

FIG. 1 is a schematic illustration showing a structure of a general IgG antibody. The IgG antibody is composed of heavy chains, each consisting of VH, CH1, hinge, CH2, and CH3, and light chains, each consisting of VL and CL.

FIG. 2A shows binding activity to human FcγRI for IgG1 WT obtained by replacing the IgG2 Fc region of the anti-EGFR antibody, panitumumab, with a human wildtype IgG1 Fc region; a variant obtained by introducing L234A/L235A (LALA) mutations into the Fc region of IgG1 WT; a variant obtained by introducing L234A/L235A/D265A (LALA-DA) mutations into the Fc region of IgG1 WT; and a variant obtained by introducing L234A/L235A/P329G (LALA-PG) mutations into the Fc region of IgG1 WT, as evaluated by an SPR method.

FIG. 2B shows binding activity for the antibodies shown in FIG. 2A to human FcγRIIa, as evaluated by an SPR method.

FIG. 2C shows binding activity for the antibodies shown in FIG. 2A to human FcγRIIb/c, as evaluated by an SPR method.

FIG. 2D shows binding activity for the antibodies shown in FIG. 2A to human FcγRIIIa, as evaluated by an SPR method.

FIG. 2E shows binding activity for the antibodies shown in FIG. 2A to human FcγRIIIb, as evaluated by an SPR method.

FIG. 3 shows binding activity for the antibodies shown in FIG. 2A to cynomolgus FcγRI, as evaluated by an SPR method.

FIG. 4A shows a change in temperature-dependent heat capacity of IgG1 WT of the anti-EGFR antibody, panitumumab, as measured by DSC. The number near each peak represents the temperature (Tₘ) at the top of the peak.

FIG. 4B shows a change in temperature-dependent heat capacity of the LALA variant of the anti-EGFR antibody, panitumumab, as measured by DSC. The number near each peak represents the Tₘ.

FIG. 4C shows a change in temperature-dependent heat capacity of the LALA-DA variant of the anti-EGFR antibody, panitumumab, as measured by DSC. The number near each peak represents the Tₘ.

FIG. 4D shows a change in temperature-dependent heat capacity of the LALA-PG variant of the anti-EGFR antibody, panitumumab, as measured by DSC. The number near each peak represents the Tₘ.

FIG. 5 shows binding activity to human FcγRI for a variant obtained by introducing L234A/L235A/D265A (LALA-DA) mutations into the Fc region of an IgG1 WT, wherein the IgG1 WT is obtained by replacing the IgG2 Fc region of an anti-EGFR antibody, panitumumab, with a human wildtype IgG1 Fc region; a variant obtained by introducing L234A/L235A/D265G (LALA-DG) mutations into the Fc region of IgG1 WT; a variant obtained by introducing L234A/L235A/D265N (LALA-DN) mutations into the Fc region of IgG1 WT; a variant obtained by introducing L234A/L235A/D265Q (LALA-DQ) mutations into the Fc region of IgG1 WT; and a variant obtained by introducing L234A/L235A/D265T (LALA-DT) mutations into the Fc region of IgG1 WT, as evaluated by an SPR method.

FIG. 6 shows binding activity for the antibodies shown in FIG. 5A to cynomolgus FcγRI, as evaluated by an SPR method.

FIG. 7A shows a change in temperature-dependent heat capacity of the LALA-DG variant of the anti-EGFR antibody, panitumumab, as measured by DSC. The number near each peak represents the Tₘ.

FIG. 7B shows a change in temperature-dependent heat capacity of the LALA-DN variant of the anti-EGFR antibody, panitumumab, as measured by DSC. The number near each peak represents the Tₘ.

FIG. 7C shows a change in temperature-dependent heat capacity of the LALA-DQ variant of the anti-EGFR antibody, panitumumab, as measured by DSC. The number near each peak represents the Tₘ.

FIG. 7D shows a change in temperature-dependent heat capacity of the LALA-DT variant of the anti-EGFR antibody, panitumumab, as measured by DSC. The number near each peak represents the Tₘ.

FIG. 8 shows time-dependent changes in blood concentrations of IgG1 WT, LALA variant, and LALA-DG variant of the anti-EGFR antibody, panitumumab, in mice after intravenous (i.v.) administration. Error bar in the figure indicates the standard deviation (n=3).

FIG. 9A shows binding activity to human FcγRI for the LALA-DG variant of the anti-EGFR antibody, panitumumab, and a variant obtained by introducing L234A/L235A/D265G/P329A (LALA-DGPA) mutations into the Fc region of IgG1 WT, as evaluated by an SPR method.

FIG. 9B shows binding activity for the antibodies shown in FIG. 9A to cynomolgus FcγRI, as evaluated by an SPR method.

FIG. 10A shows binding activity to human FcγRI for a LALA variant and a LALA-DG variant of the anti-CD70 antibody, vorsetuzumab, which is an IgG1 antibody, as evaluated by an SPR method.

FIG. 10B shows binding activity to human FcγRI for a LALA variant and a LALA-DG variant of the anti-LPS antibody, O11-1111, which is an IgG1 antibody, as evaluated by an SPR method.

FIG. 11A presents the amino acid sequence of wildtype human IgG1 (G1m17) constant region, SEQ ID No: 1. Underlined, double-underlined and boxed portions show the hinge region, CH2 and CH3 domains, and positions for amino acid substitutions (mutations), respectively.

FIG. 11B presents the amino acid sequence of wildtype human IgG1 (G1m3) constant region, SEQ ID No: 2. Notations as for FIG. 11A.

FIG. 11C represents the amino acid sequence of wildtype human IgG4 constant region, SEQ ID No: 3. Notations as for FIG. 11A.

FIG. 12A presents the amino acid sequence of the LALA-DG variant of human IgG1 (G1m17) constant region, SEQ ID No: 4. Notations as for FIG. 11A.

FIG. 12B presents the amino acid sequence of the LALA-DG variant of human IgG1 (G1m3) constant region, SEQ ID No: 5. Notations as for FIG. 11A.

FIG. 13A presents the amino acid sequence of the LALA-DN variant of human IgG1 (G1m17) constant region, SEQ ID No: 6. Notations as for FIG. 11A.

FIG. 13B presents the amino acid sequence of the LALA-DN variant of human IgG1 (G1m3) constant region, SEQ ID No: 7. Notations as for FIG. 11A.

FIG. 14A presents the amino acid sequence of the LALA-DGPA variant of human IgG1 (G1m17) constant region, SEQ ID No: 8. Notations as for FIG. 11A.

FIG. 14B presents the amino acid sequence of the LALA-DGPA variant of human IgG1 (G1m3) constant region, SEQ ID No: 9. Notations as for FIG. 11A.

FIG. 15A presents the amino acid sequence of the FALA-DG variant of human IgG4 constant region, SEQ ID No: 10. Notations as for FIG. 11A.

FIG. 15B presents the amino acid sequence of the FALA-DN variant of human IgG4 constant region, SEQ ID No: 11. Notations as for FIG. 11A.

FIG. 15C presents the amino acid sequence of the FALA-DGPA variant of human IgG4 constant region, SEQ ID No: 12. Notations as for FIG. 11A.

FIG. 16A presents the amino acid sequence of the IgG1 WT light chain of the anti-EGFR antibody, panitumumab, SEQ ID No: 13. Underlined portion shows the variable region.

FIG. 16B presents the amino acid sequence of the IgG1 WT heavy chain of the anti-EGFR antibody, panitumumab, SEQ ID No: 14. Underlined, double-underlined and boxed portions show the hinge region, CH2 and CH3 domains, and positions for amino acid substitutions (mutations), respectively.

FIG. 17A presents the amino acid sequence of the light chain of a LALA variant of the anti-CD70 antibody, vorsetuzumab, SEQ ID No: 15. Underlined portion shows the variable region.

FIG. 17B presents the amino acid sequence of the heavy chain of a LALA variant of the anti-CD70 antibody, vorsetuzumab, SEQ ID No: 16. Notations as for FIG. 16B.

FIG. 18A presents the amino acid sequence of the IgG1 WT light chain of the anti-LPS antibody, O11-1111, SEQ ID No: 17. Underlined portion shows the variable region.

FIG. 18B presents the amino acid sequence of the IgG1 WT heavy chain of the anti-LPS antibody, O11-1111, SEQ ID No: 18. Notations as for FIG. 16B.

FIG. 19A presents the amino acid sequence of human Fc gamma RI (Accession #: P12314), SEQ ID No: 19. Underlined portion shows the sequence of Recombinant Human Fc gamma RI/CD64 Protein, CF (R&D Systems).

FIG. 19B presents the amino acid sequence of human Fc gamma RIIa (R167) (Accession #: AAA35827), SEQ ID No: 20. Underlined portion shows the sequence of Recombinant Human Fc gamma RIIa/CD32a (R167) Protein (R&D Systems).

FIG. 19C presents the amino acid sequence of human Fc gamma RIIa (H167) (Accession #: P12318-1 (P12318.4)), SEQ ID No: 21. Underlined portion shows the sequence of Recombinant Human Fc gamma RIIa/CD32a (H167) Protein (R&D Systems).

FIG. 19D presents the amino acid sequence of human Fc gamma RIIb/c (Accession #: P31994), SEQ ID No: 22. Underlined portion shows the sequence of Recombinant Human Fc gamma RIIB/C (CD32b/c) Protein, CF (R&D Systems).

FIG. 19E presents the amino acid sequence of human Fc gamma RIIIa (Accession #: AAH17865), SEQ ID No: 23. Underlined portion shows the sequence of Recombinant Human Fc gamma RIIIa/CD16a Protein, CF (R&D Systems).

FIG. 19F presents the amino acid sequence of human Fc gamma RIIIb (Accession #: O75015), SEQ ID No: 24. Underlined portion shows the sequence of Recombinant Human Fc gamma RIIIB/CD16b Protein (R&D Systems).

FIG. 20A presents the amino acid sequence of cynomolgus Fc gamma RI (Accession #: NP_001270969), SEQ ID No: 25. Underlined portion shows the sequence of Recombinant Cynomolgus Monkey Fc gamma RI/CD64 Protein, CF (R&D Systems).

FIG. 20B presents the amino acid sequence of cynomolgus Fc gamma RIIa (Accession #: NP_001270598), SEQ ID No: 26. Underlined portion shows the sequence of Recombinant Cynomolgus Monkey Fc gamma RIIa/CD32a Protein, CF (R&D Systems).

FIG. 20C presents the amino acid sequence of cynomolgus Fc gamma RIIb (Accession #: NP_001271060), SEQ ID No: 27. Underlined portion shows the sequence of Recombinant Cynomolgus Monkey Fc gamma RIIB Protein, CF (R&D Systems).

FIG. 20D presents the amino acid sequence of cynomolgus Fc gamma RIII (Accession #: NP_001270121), SEQ ID No: 28. Underlined portion shows the sequence of Recombinant Cynomolgus Monkey Fc gamma RIII/CD16 Protein, CF (R&D Systems).

FIG. 21 presents the amino acid sequence of human neonatal Fc receptor large subunit p51 (Accession #: P55899), SEQ ID No: 29.

FIG. 22 presents the amino acid sequence of beta2-microglobulin (Accession #: NP_004039.1), SEQ ID No: 30.

FIG. 23 presents the amino acid sequence of human Fc gamma RIIIa (V176F) (Accession #: P08637), SEQ ID No: 31. Underlined portion shows the sequence of Recombinant Human Fc gamma RIIA/CD16a (V176F) Protein (R & D Systems).

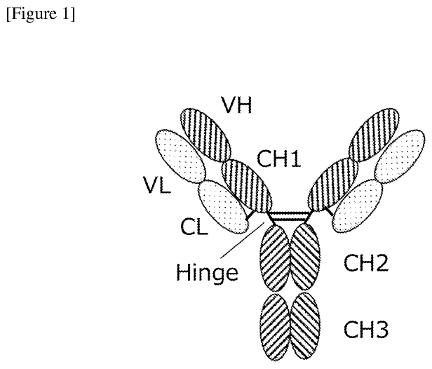

### DETAILED DESCRIPTION

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method, use or composition disclosed herein, and vice versa.

The present disclosure relates to a polypeptide comprising one or more variant IgG1 Fc regions or variant IgG4 Fc regions, wherein each of the variant Fc regions comprises two CH2 domains, wherein one or both of the CH2 domains in at least one of the variant Fc regions is/arc variant CH2 domain(s), and wherein amino acid residues at positions 234, 235, and 265 (all positions expressed by EU numbering) of the variant CH2 domain are Ala, Ala, and Gly, respectively (and optionally, an amino acid residue at position 329 is Ala), or are Ala, Ala, and Asn, respectively (and optionally, an amino acid residue at position 329 is Ala). The disclosure further relates to a molecule comprising said polypeptide; a polypeptide-drug conjugate in which said polypeptide is conjugated to a drug; a pharmaceutical composition comprising said polypeptide, molecule or conjugate; and a method for treating a human subject with said polypeptide, molecule, conjugate, or pharmaceutical composition. In certain embodiments, the disclosure relates to a polypeptide comprising a variant IgG Fc region, a molecule comprising said polypeptide, and a polypeptide-drug conjugate in which said polypeptide is conjugated to a drug, which have reduced binding activity to Fcγ receptors and good thermal stability and are useful as a medicament. For example, in one embodiment, the polypeptide comprising a variant CH2 domain, the molecule comprising the polypeptide, and the polypeptide-drug conjugate in which the polypeptide is conjugated to a drug have reduced binding activity to an Fcγ receptor compared to a parent polypeptide, molecule and conjugate, respectively, comprising a parent CH2 domain (i.e. a CH2 domain prior to introducing the mutations at positions 234, 235 and 265 (and optionally position 329) comprised by the variant CH2 domain). In another embodiment, the polypeptide, the molecule and the conjugate have improved or equivalent thermal stability compared to a parent polypeptide, molecule and conjugate, respectively, comprising a parent CH2 domain. In another embodiment, the polypeptide, the molecule and the conjugate maintain binding activity to an antigen and have no decrease in half-life in blood compared to a parent polypeptide, molecule and conjugate, respectively, comprising a parent CH2 domain. In another embodiment, the CH2 domain of the polypeptide, molecule or conjugate has a small number of amino acid substitutions (for example, 3 or 4 amino acid substitutions) from a parent polypeptide, molecule and conjugate, respectively, comprising a parent CH2 domain which lowers the immunogenicity risk that may occur with an increased number of amino acid substitutions.

As used herein, the numbering of amino acid residues in the Fc region is according to the EU index (Proceedings of the National Academy of Sciences of the United States of America, Vol. 63, No. 1 (May 15, 1969), pp. 78-85) unless otherwise stated.

As used herein, “polypeptide” means a molecule typically comprising about 10 or more amino acid residues which are connected to each other by peptide bonds. A polypeptide according to the present disclosure may be derived from a natural polypeptide or a synthetic or recombinant polypeptide. The polypeptide according to the present disclosure may be, for example, an antibody or a functional fragment thereof, a fusion protein, or the like, as described herein.

In one embodiment, the present disclosure relates to a molecule comprising the above-mentioned polypeptide. Such molecules include, but are not limited to, bispecific antibodies, multispecific antibodies, soluble receptors, immunocytokines, moieties that bind to antigens or targets other than antibodies or functional fragments thereof (for example, non-immunoglobulin proteins, receptors, ligands, nucleic acid aptamers, anti-sense nucleic acid molecules, low molecular weight compounds, and the like). When a molecule comprising the polypeptide according to the present disclosure is itself a polypeptide, the molecule may be regarded as a “polypeptide” according to the present disclosure, and thereby may be referred to herein either as a “polypeptide according to the present disclosure” or as a “molecule comprising the polypeptide according to the present disclosure.” A “fusion protein” as described below may also in some embodiments be itself a polypeptide, and thus may be referred to herein either as a “polypeptide according to the present disclosure” or as a “molecule comprising the polypeptide according to the present disclosure.”

In one embodiment, the present disclosure relates to a polypeptide-drug conjugate, in which the polypeptide described above is conjugated to a drug. The drug can be, for example, a therapeutic drug or a diagnostic drug. When the polypeptide comprised by the polypeptide-drug conjugate is an antibody that targets tumor cells, it is preferable, but not essential, that the antibody itself has an antitumor effect. A polypeptide-drug conjugate may usually be prepared by conjugating the polypeptide to the drug via a linker. Linkers known in the art, for example, a sugar linker and/or a peptide linker, may be used. In addition, various pharmaceutical agents may be used as the drug, depending on the purpose of intended therapy in which the polypeptide-drug conjugate will be used. A polypeptide-drug conjugate, in which the polypeptide described above is conjugated to a drug, may be regarded as one of the other embodiments of a “molecule comprising the polypeptide according to the present disclosure.”

### Variant Fc Region

The polypeptide according to the present disclosure comprises a variant IgG1 Fc region, a variant IgG4 Fc region or a variant IgG1/IgG4 region (collectively a “variant Fc region”). The “Fc region” as used herein, refers to a C-terminal region in a heavy chain of an antibody and comprises two CH2 domains and two CH3 domains. The Fc region is usually in the form of a homo- or hetero-dimer of the C-terminal regions connected by a hinge region in two heavy chains, but may also be a single-stranded Fc (scFc) region in some embodiments. When the Fc region is derived from an IgG1 antibody, the Fc region generally means, but is not limited to, the region from the amino acid residue at position 231 to the C-terminus.

As used herein, the term “hinge” or “hinge region” refers to a part of the IgG antibody comprising the C-terminus of a CH1 domain to an N-terminus of the CH2 domain. When the Fc region is derived from an IgG1 or IgG4 antibody, the hinge generally extends, but without limitation, from an amino acid residue at about position 216 to an amino acid residue at about position 230. As described above, when the Fc region is derived from the IgG1 antibody, the Fc region generally refers to the part from an amino acid residue at position 231 to the C-terminus of the heavy chain, however, in some embodiments, the “hinge” may be included in the Fc region.

As used herein, a “CH2 domain” includes, but is not limited to, the region extending from a point connecting with the hinge to a point connecting with the CH3 domain. When the Fc region is derived from an IgG1 or IgG4 antibody, the CH2 domain generally extends from an amino acid residue at about position 231 to an amino acid residue at about position 340. The amino acid residues at positions 234, 235, 265, and 329 in the Fc-region of human wild-type IgG1 or IgG4 are present in the CH2 domain. The CH2 domain usually has a looped structure in the Fc-region, but is not limited to such a structure.

As used herein, a “CH3 domain” includes, but is not limited to, the region extending from a point connecting with the CH2 domain of the Fc region to the C-terminus of the heavy chain. When the Fc region is derived from an IgG1 or IgG4 antibody, the CH3 domain generally extends, but is not limited to, from the amino acid residue at about position 341 to the amino acid residue at about position 447 of IgG1. The CH3 domain usually has a looped structure in the Fc-region, but is not limited to such a structure.

As used herein, a “variant IgG1 Fc region” or a “variant IgG4 Fc region” refer to an IgG1 Fc region or an IgG4 Fc region in which one or both of the two CH2 domains is/are variant CH2 domain(s). Variant IgG1 Fc regions and variant IgG4 Fc regions may collectively be referred to herein as “variant Fc regions.” The term “variant Fc region” also encompasses a mixed IgG1/IgG4 Fc region in which one or both of the two CH2 domains is/are variant CH2 domain(s). A “variant CH2 domain” refers to a CH2 domain in which amino acid residues at positions 234, 235, and 265 (all positions expressed by EU numbering) are Ala, Ala, and Gly; or Ala, Ala, and Asn, respectively (i.e., 234A, 235A, and 265G; or 234A, 235A, and 265N). In one embodiment, the variant CH2 domain comprises amino acid residues at positions 234, 235, 265, and 329 substituted by Ala, Ala, Gly, and Ala, respectively (i.e., 234A, 235A, 265G, and 329A).

The polypeptide according to the present disclosure may comprise another (one or more) component(s) other than the variant Fc region, such as a portion, region, or domain of any one of the IgG subclasses: IgG1, IgG2, IgG3, or IgG4. The Fc region is preferably derived from a mammal. In some embodiments, the Fc region is derived from a human or cynomolgus monkey (including, for example, crab-eating macaques, rhesus monkeys, common marmosets, squirrel monkeys, and the like). In some embodiments, the Fc region is derived from a human.

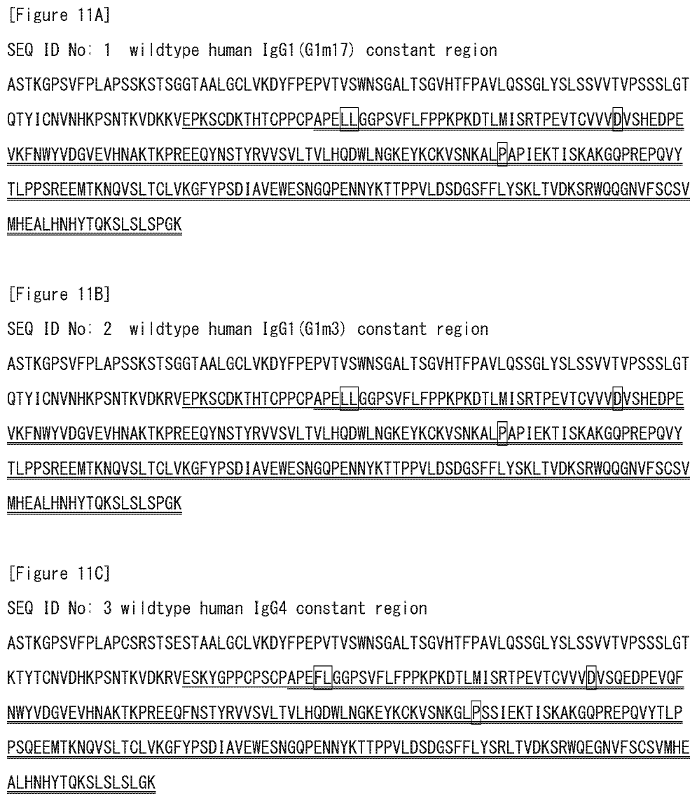

Examples of amino acid sequences of an Fc region of wildtype human IgG1 are provided herein as the amino acid sequence consisting of the 114th to 330th amino acids of SEQ ID No: 1 or 2 (see FIGS. 11A and 11B), and an example of the amino acid sequence of an Fc region of wildtype human IgG4 is provided herein as the amino acid sequence consisting of the 111th to 327th amino acids of SEQ ID No: 3 (see FIG. 11C). Amino acid residues at positions 234, 235, 265, and 329 (all positions expressed by EU numbering) in a human IgG1 Fc region prior to introducing the mutations by the above-mentioned amino acid substitutions are Leu, Leu, Asp, and Pro, respectively (i.e., L234, L235, D265 and P329). The above-mentioned amino acid substitutions may thus be represented herein as L234A/L235A/D265G (LALA-DG), L234A/L235A/D265G/P329A (LALA-DGPA), and L234A/L235A/D265N (LALA-DN), and a polypeptide comprising those amino acid substitutions may be referred herein as “LALA-DG variant”, “LALA-DGPA variant” and “LALA-DN variant”, respectively. The amino acid residues at positions 234, 235, 265, and 329 (all positions expressed by EU numbering) in the human IgG4 Fc region prior to introducing the mutations are of the same as the IgG1 Fc region, except that the amino acid residue at position 234 is Phe (i.e., F234, L235, D265, and P329). When the “CH2 domain according to the present disclosure” is derived from an IgG4 antibody, the above-mentioned amino acid substitutions may thus be represented herein as F234A/L235A/D265G (FALA-DG), F234A/L235A/D265G/P329A (FALA-DGPA), and F234A/L235A/D265N (FALA-DN), and a polypeptide comprising those amino acid substitutions may thus be referred herein as “FALA-DG variant”, “FALA-DGPA variant” and “FALA-DN variant”, respectively.

A “variant CH2 domain” according to the present disclosure comprises the above-mentioned specific amino acid substitutions at positions 234, 235, and 265 (and optionally at position 329), and may further include amino acid modification(s) (for example, deletion, addition, substitution, or insertion) other than the above-mentioned specific amino acid substitutions. Similarly, a variant Fc region may comprise amino acid modification(s) (for example, deletion, addition, substitution, or insertion) other than the above-mentioned specific amino acid substitutions. When the Fc region comprises additional amino acid modifications, the additional amino acid modifications may be in the CH2 domain, the CH3 domain, the hinge region (when included) or a combination thereof. As noted above, the Fc region comprises two CH2 domains and two CH3 domains. When the Fc region comprises additional amino acid modifications, the modifications may be symmetric modifications (the same modifications present in both CH2 domains and/or both CH3 domains) or they may be asymmetric modifications (different modifications in each CH2 domain and/or CH3 domain, or present in just one of the CH2 domains and/or one of the CH3 domains).

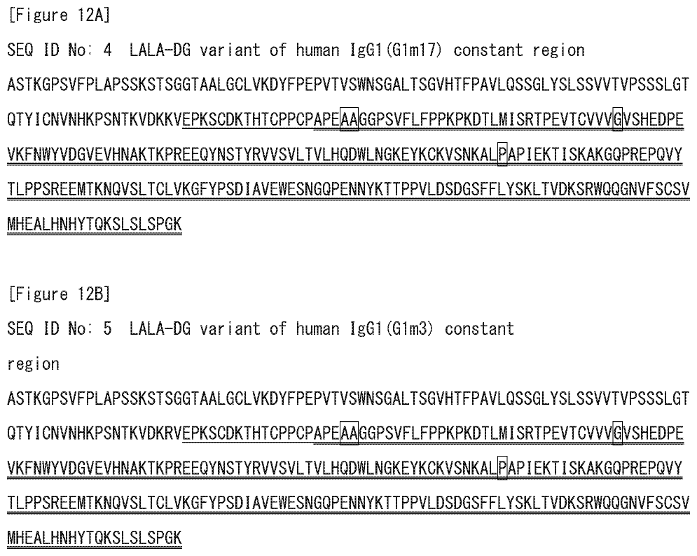

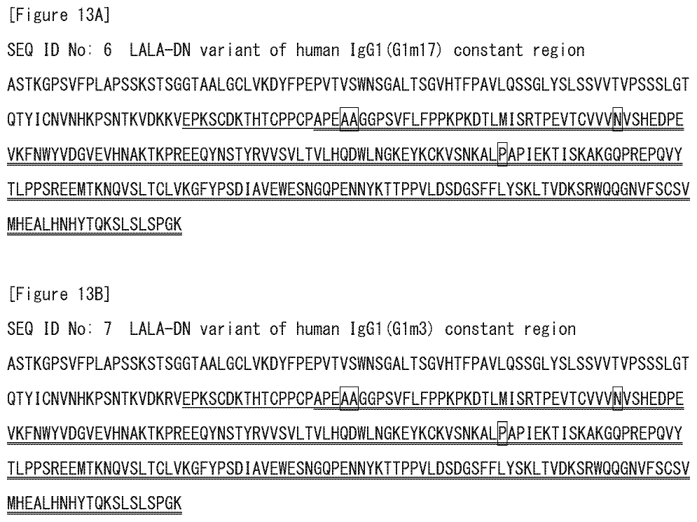

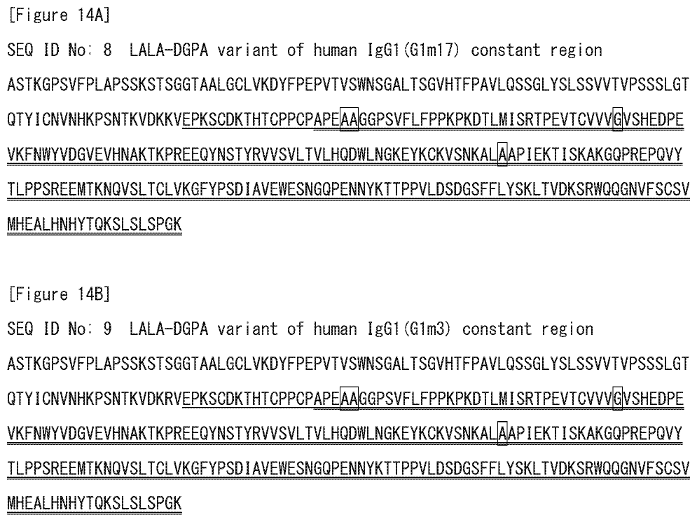

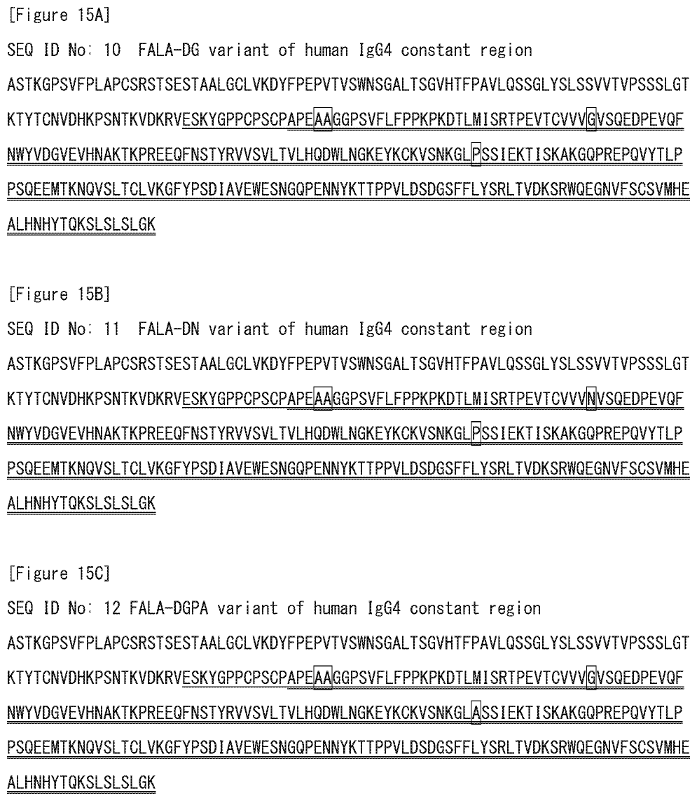

Examples of amino acid sequences of a LALA-DG variant of a human IgG1 Fc region are provided herein as the amino acid sequence consisting of the 114th to 330th amino acids of SEQ ID No: 4 or 5 (see FIGS. 12A and 12B), examples of amino acid sequences of a LALA-DN variant of a human IgG1 Fc region are provided herein as the amino acid sequence consisting of the 114th to 330th amino acids of SEQ ID No: 6 or 7 (see FIGS. 13A and 13B), and examples of amino acid sequences of a LALA-DGPA variant of a human IgG1 Fc region are provided herein as the amino acid sequence consisting of the 114th to 330th amino acids of SEQ ID No: 8 or 9 (see FIGS. 14A and 14B). An example of an amino acid sequence of a FALA-DG variant of a human IgG4 Fc region is provided herein as the amino acid sequence consisting of the 111th to 327th amino acids of SEQ ID No: 10 (see FIG. 15A), an example of an amino acid sequence of a FALA-DN variant of a human IgG4 Fc region is provided herein as the amino acid sequence consisting of the 111th to 327th amino acids of SEQ ID No: 11 (see FIG. 15B), and an example of an amino acid sequence of a FALA-DGPA variant of a human IgG4 Fc region is provided herein as the amino acid sequence consisting of the 111th to 327th amino acids of SEQ ID No: 12 (see FIG. 15C). It is to be understood that these sequences are provided as examples only and the variant CH2 domains of the present disclosure are not limited thereto. In addition, it is to be understood that a “LALA-DNPA variant” and a “FALA-DNPA variant” in which the 265th Gly of the “LALA-DGPA variant” or “FALA-DGPA variant,” respectively, is replaced with Asn are also encompassed by the present disclosure.

A CH2 domain prior to introducing the above-mentioned specific amino acid substitutions (mutations) at positions 234, 235, and 265 (and optionally at position 329) is referred herein to as a “parent CH2 domain.” The parent CH2 domain may be, but is not limited to, a wildtype CH2 domain of a IgG1 antibody or a IgG4 antibody. For example, if amino acid modification(s) have already been included in the CH2 domain prior to introducing the above-mentioned specific amino acid substitutions, then the “parent CH2 domain” means a CH2 domain comprising such previous modification(s).

Likewise, an Fc region comprising the above-mentioned “parent CH2 domain” is referred to herein as a “parent Fc region” and a polypeptide comprising the above-mentioned “parent CH2 domain” or “parent Fc region” is referred to herein as a “parent polypeptide.” The parent polypeptide typically refers to, but is not limited to, a polypeptide that is identical or equivalent to a polypeptide according to the present disclosure, except that the parent polypeptide comprises a parent CH2 domain in place of the variant CH2 domain of the present disclosure.

A polypeptide according to the present disclosure comprises the variant Fc region in which the above-mentioned specific amino acid substitutions have been introduced into the parent CH2 domain(s). As described above, since two CH2 domains are present in one Fc region, the amino acid substitutions in the variant Fc region may be introduced into only one of the two parent CH2 domains or may be introduced into both parent CH2 domains. In addition, when amino acid substitutions are introduced into both parent CH2 domains, the same or different amino acid substitutions may be introduced. For example, one of the two variant CH2 domains in the variant Fc region may comprise the amino acid substitutions L234A/L235A/D265G, and the other may comprise the amino acid substitutions L234A/L235A/D265N. Alternatively, one of the two variant CH2 domains in the variant Fc region may comprise the amino acid substitutions L234A/L235A/D265G, and the other may comprise the amino acid substitutions L234A/L235A/D265G/P329A. Other combinations are also possible and encompassed by the present disclosure. When the same amino acid substitutions are introduced into the two CH2 domains of the Fc region, the domains and the Fc region may be referred to as a “homodimer,” which is formed by “homodimerization”, and when different amino acid substitutions are introduced into the two CH2 domains of the Fc region, the domains and the Fc region may be referred to as a “heterodimer,” which is formed by “heterodimerization”. “Heterodimerization” can be achieved using various known techniques, such as the “knobs-into-holes” technique (International Publication No. WO 96/027011), “electrostatic steering” (J Biol Chem, 285:19637-46 (2010)), strand exchange engineered domain (SEED) technology (Prot Eng Des Sel, 23(4):195-202 (2010)), Fab-arm exchange (Proc Natl Acad Sci USA, 110(13):5145-50 (2013)) and approaches combining positive and negative design strategies to produce stable asymmetrically modified Fc regions as described in International Publication Nos. WO 2012/058768 and WO 2013/063702. Further, when the polypeptide of the present disclosure comprises a plurality of antibodies, at least one of the antibodies should comprise at least one variant CH2 domain. Moreover, in some embodiments, one of the two CH2 domains of the variant Fc region may be a CH2 domain derived from an IgG1 antibody and the other may be a CH2 domain derived from an IgG4 antibody. Alternatively, in some embodiments, both CH2 domains of a variant Fc region may be derived from an IgG1 antibody or both may be derived from an IgG4 antibody.

In one embodiment, the variant CH2 domain in which the amino acid substitutions LALA-DG, LALA-DN or LALA-DGPA have been introduced into the parent CH2 domain has improved thermal stability compared to the parent CH2 domain, i.e., the CH2 domain prior to introducing the above-mentioned specific amino acid substitutions at positions 234, 235, and 265 (and optionally at position 329). In a further embodiment, the variant CH2 domain has equivalent or improved thermal stability compared to the parent CH2 domain. In another embodiment, the variant CH2 domain in which the amino acid residues at positions 234, 235 and 265 (all positions expressed by EU numbering) are Ala, Ala and Gly, respectively, and the variant CH2 domain in which the amino acid residues at positions 234, 235, 265, and 329 (all positions expressed by EU numbering) are Ala, Ala, Gly, and Ala, respectively, have equivalent thermal stability compared to their respective parent CH2 domains. In another embodiment, the variant CH2 domain in which the amino acid residues at positions 234, 235 and 265 (all positions expressed by EU numbering) are Ala, Ala and Asn, respectively, has equivalent thermal stability compared to the parent CH2 domain in which the amino acid residues at positions 234, 235 and 265 (all positions expressed by EU numbering) are Leu, Leu and Asp, respectively.

The term “thermal stability” as used herein is determined by a thermal denaturation midpoint temperature (Tₘ) of the CH2 domain of the Fc region. The thermal denaturation midpoint temperature (Tm) may be measured, for example, by DSC (differential scanning calorimetry) or DSF (differential scanning fluorometry).

In one embodiment, the thermal denaturation midpoint temperature (Tm) may be determined by DSC measurement in which a change in heat capacity is observed with increasing temperature. For example, in the measurement of the thermal denaturation midpoint temperature (Tm) of a polypeptide according to the present disclosure, an evaluation of the thermal stability of the CH2 domain may be conducted by making an IgG antibody, that is, an antibody comprising a Fc region (i.e., a dimer comprising a hinge portion, a CH2 domain, and a CH3 domain) and a Fab region, and then assessing the thermal stability of the antibody by DSC (see, for example, the Examples section herein). When DSC analysis is conducted for an IgG antibody, in general, peaks for thermal denaturation in the order of CH2 domain, Fab region, and CH3 domain are observed with increasing temperature. In the present disclosure, the thermal denaturation midpoint temperature (Tₘ) was determined by peak temperature for thermal denaturation and used to evaluate thermal stability.

An example of a specific procedure for measurement of Tm by DSC is as follows: first, a solution of the polypeptide according to the present disclosure is prepared in histidine buffer, citrate buffer, TBS, or the like (generally, at 0.1 μg/mL to 100 μg/mL), and then the solution of the polypeptide is placed in a measurement pan of a DSC instrument. Subsequently, the temperature is raised at a constant temperature rise rate to observe an endothermic peak for the CH2 domain, based on which the Tₘ value is determined. The DSC measurement of the parent polypeptide may be conducted in a similar manner to determine the Tm of the parent polypeptide, and the difference (ΔTₘ) in the Tₘ values between the polypeptide of the present disclosure and the parent polypeptide may then be determined. An instrument for DSC measurement commonly used in the art, such as MicroCal VP-Capillary DSC System (Malvern Panalytical Ltd., Malvern, UK), may be used. In one embodiment, the thermal stability of a polypeptide may be determined utilizing methods described in Examples 5, 11 or 14 herein.

As used herein, “improved thermal stability” means that the thermal denaturation midpoint temperature (Tₘ) of the variant CH2 domain is higher by 1° C. or more than the thermal denaturation midpoint temperature (Tₘ) of a reference CH2 domain (for example, the parent CH2 domain). As used herein, “equivalent thermal stability” means that the thermal denaturation midpoint temperature (Tₘ) of the variant CH2 domain is not higher by 1° C. and is not lower by 1° C. (i.e., is within (±) 1° C.) than that of a reference CH2 domain (for example, the parent CH2 domain). In one embodiment, when the variant CH2 domain is a LALA-DG variant, the Tₘ is 3° C. or higher or 4° C. or higher than that of the CH2 domain of the wild-type Fc region, and the Tₘ is 3° C. or more higher than that of the CH2 domain of a LALA variant (a variant in which amino acid residues at positions 234 and 235 (all positions expressed by EU numbering) in the human IgG1 Fc region are Ala and Ala, respectively). Thus, in one embodiment, the thermal stability of the LALA-DG variant is improved over that of both the wild-type CH2 domain and also the CH2 domain of the LALA mutant, from which the LALA-DG variant differs by only one additional substitution at the 265 position. In one embodiment, the variant CH2 domain has a Tₘ that is about 0.1° C., 0.5° C., 1° C. or higher, than that of the parent CH2 domain. In one embodiment, the variant CH2 domain has a Tₘ that is about 1.5° C., 2° C., 2.5° C., or 3° C. or higher, than that of the parent CH2 domain. In one embodiment, the variant CH2 domain has a Tₘ that is about 3.5° C., 4° C., 4.5° C., or 5° C. or higher, than that of the parent CH2 domain. As described above, other additional amino acid modification(s) (deletion(s), addition(s), substitution(s), etc.) may be introduced in the variant CH2 domain and/or the CH3 domain in addition to the specific amino acid substitutions at positions 234, 235, and 265 (optionally, position 329). As used herein, the term “about” refers to ±10% of an indicated value.

In one embodiment, the polypeptide according to the present disclosure has a reduced or attenuated binding affinity to an Fcγ receptor compared to a parent polypeptide. In one embodiment, the polypeptide according to the present disclosure has a reduced or attenuated binding affinity to an Fcγ receptor compared to a parent polypeptide, and thereby an effector function of the polypeptide is reduced compared to the parent polypeptide. Unless otherwise specified, “binding affinity” and “binding activity” as used herein have the same meaning. The term ““Fcγ receptor”” (also referred to as an Fc gamma receptor or FcγR) refers to a receptor capable of binding to the Fc region of an IgG antibody. The FcγR may be derived from a mammal, such as human, monkey, or rat. In one embodiment, the FcγR is a human FcγR or a cynomolgus FcγR. In one embodiment, the FcγR is a human FcγR. The human receptor families include, but are not limited to, FcγRI (CD64) (including isoforms FcγRIa, FcγRIb, and FcγRIc), FcγRII (CD32) (isoforms FcγRIIa (including allotypes H131 (H type) and R131 (R type)), FcγRIIb (including FcγRIIb-1 and FcγRIIb-2), and FcγRIIc (including FcγRIIc), and FcγRIII (CD16) (isoforms FcγRIIIa (including allotypes V158 and F158), FcγRIIIb (including allotypes FcγRIIIb-NA1 and FcγRIIIb-NA2), and FcγRIIIc). The cynomolgus Fcγ receptor family includes, but is not limited to, the above FcγRI, FcγRII, and FcγRIII (including their isoforms), except for FcγRIIc and FcγRIIIb. The FcγR receptor may also include allelic variants and alternatively spliced forms of the receptors. As used herein “reduced binding affinity to FcγR” means that the activity for at least one of the nine isoforms of FcγRI, FcγRII, and FcγRIII described above may be reduced, for example, the binding affinity to at least two, at least three, at least four, at least five, at least six, at least seven, at least eight, or all FcγR isoforms may be reduced. In one embodiment, the polypeptide according to the present disclosure has reduced binding to one or more of an FcγRI, an FcγRII or an FcγRIII. In one embodiment, the polypeptide according to the present disclosure has reduced binding to an FcγRI, an FcγRII and an FcγRIII. The FcγRI, FcγRII and/or FcγRIII may be human FcγRI, FcγRII and/or FcγRIII or cynomolgus FcγRI, FcγRII and/or FcγRIII. In one embodiment, the polypeptide according to the present disclosure has reduced binding to one or more of a human FcγRI, FcγRIIa, FcγRIIb, FcγRIIIa or FcγRIIIb. In one embodiment, the polypeptide according to the present disclosure has reduced binding to a human FcγRI, FcγRIIa, FcγRIIb, FcγRIIla and FcγRIIIb. As Fcγ receptors of human and cynomolgus origin, the receptors disclosed in Comparative Examples and Examples herein, can be exemplified, but not limited thereto.

The term “effector function” as used herein refers to a biological activity attributable to, or mediated by, an Fc region of an antibody. Effector functions include, but are not limited to, complement-dependent cellular cytotoxicity (CDC), antibody-dependent cell-mediated cytotoxicity (ADCC), antibody-dependent cell-mediated phagocytosis (ADCP), Fc-receptor binding, C1q binding, downregulation of cell-surface receptors (for example, B-cell receptors), and production of cytokines. In one embodiment, the polypeptide according to the present disclosure can reduce effector function by the above-mentioned specific amino acid substitutions at positions 234, 235, and 265 (and optionally at position 329), where the effector function is one or more of CDC activity, ADCC activity, ADCP activity, Fc-receptor binding, C1q binding, downregulation of cell-surface receptors, or excessive cytokine production, and may be useful for pharmaceutical applications in which effector function is unnecessary. Amino acid modifications that reduce effector function may be referred to herein as “effectorless mutations.” The effector function can be assessed using assays known in the art, as well as measurements of binding affinity to the Fcγ receptor such as those disclosed herein.

The “binding affinity to an Fcγ receptor” can be measured by a known method, for example, it can be measured by surface plasmon resonance (SPR) methods in which an interaction between a ligand and an analyte is measured. This measurement can be conducted, for example, using a Biacore™ SPR system (Cytiva, Marlborough, MA). More specifically, in this method, each His tagged FcγR is immobilized as a ligand on the sensor chip directly or by capture with anti-His tag antibodies on the sensor chip, and then the polypeptide of the present disclosure is added to the sensor chip as an analyte. When the added analyte binds to the immobilized ligand, the apparent mass of the immobilized ligand molecule increases. The amount of the position shift of the SPR signal is measured, which depends on the change in the refractive index of the solvent on the sensor chip surface. Based on the amount of the shift, an amount of the analyte (antibody) bound to the ligand (FcγR) is determined as a value for binding response (Resonance Unit, RU) (see Proc. Natl. Acad. Sci USA, 103(11):4005-4010 (2006), and the like). Alternatively or in addition, the binding affinity to the Fcγ receptor can be measured by ELISA, FACS, and the like. In one embodiment, the “binding affinity to an Fcγ receptor” can be determined using methods described in Examples 1-4, 9-10, 12 or 13 herein.

As used herein, “reduced or attenuated binding affinity to an Fcγ receptor” means that the binding affinity of a polypeptide according to the present disclosure to an Fcγ receptor is lower than the binding affinity of a reference polypeptide (for example, a parent polypeptide) to the same Fcγ receptor, as determined by the same assay. Reduced or attenuated binding affinity may be determined, for example, by measuring whether the RU value for a polypeptide according to the present disclosure for at least one Fcγ receptor as determined by the above-described surface plasmon resonance assay is reduced compared to the RU value of a reference polypeptide (for example, a parent polypeptide) for the same Fcγ receptor as determined by the same assay. In one embodiment, in order to effectively reduce the effector function, when the binding affinity to the Fcγ receptor of the parent polypeptide comprising the parent CH2 domain is considered as 100%, the binding affinity of the polypeptide according to the present disclosure to the same Fcγ receptor is reduced to below 80%, below 70% or below 60%. In one embodiment, when the binding affinity to the Fcγ receptor of the parent polypeptide comprising the parent CH2 domain is considered as 100%, the binding affinity of the polypeptide according to the present disclosure to the same Fcγ receptor is reduced to below 50%, below 40%, below 30%, below 20% or below 10%. In one embodiment, when the binding affinity to the Fcγ receptor of the parent polypeptide comprising the parent CH2 domain is considered as 100%, the binding affinity of the polypeptide according to the present disclosure to the same Fcγ receptor is reduced to below 5%, below 4%, below 3%, below 2%, below 1% or below 0.1%.

As is known in the art, amino acid substitutions in various region of an antibody (including the Fc region) may result in the antibody having reduced antigen-binding activity. In one embodiment, the polypeptide according to the present disclosure does not show significantly reduced “antigen-binding activity” compared to the parent polypeptide. Antigen-binding activity of the polypeptide can be measured by a known method, for example, SPR method (see, for example, the method described in Example 6 herein).

As is also known in the art, amino acid substitutions in various regions of an antibody (including the Fc region) may result in the antibody having a reduced half-life in blood. In one embodiment, the polypeptide according to the present disclosure does not show significantly reduced “blood half-life” as compared to the parent polypeptide. Blood half-life of the polypeptide can be determined by a known method, for example, using the methods described in Example 8 herein. Blood half-life of an antibody or functional fragments thereof can be extended by chemical modification, for example, by covalently binding to a polymer.

As is also known in the art, binding activity of an antibody to the neonatal Fc receptor, FcRn, may be reduced by amino acid substitutions in various regions (including the Fc region) of the antibody. In one embodiment, in relation to the above-mentioned blood half-life, the polypeptide of the present disclosure does not show significantly reduced “binding activity to neonatal Fc receptor (FcRn)” as compared to the parent polypeptide. As is known in the art, an antibody, when administered, is non-specifically taken up by vascular endothelial cells, and the like, at a constant rate by pinocytosis. The antibody binds to FcRn in endosomes at a low pH (about pH 6.0) and is transported to the outside of the cell via FcRn, whereby lysosomal translocation and degradation of the antibody can be avoided. Binding of an antibody to FcRn thus contributes to a long half-life of the antibody in blood. Binding activity of the polypeptide to FcRn can be measured by a known method, for example, a Bio-Layer Interferometry method (see, for example, the method described in Example 7 herein).

### Antibodies and Functional Fragments Thereof

In one embodiment, the polypeptide according to the present disclosure may be an antibody or a fragment thereof (for example, a functional fragment). An “antibody” is a molecule (immunoglobulin) that comprises an antigen-binding site that immuno-specifically binds an antigen, and generally comprises a heavy chain variable region, a heavy chain constant region, a light chain variable region, and a light chain constant region. An “antibody” may be any one of a polyclonal antibody, a bispecific antibody, a multispecific antibody, a chimeric antibody, a humanized antibody, a human antibody, functional fragments thereof, or modifiers thereof. The antibody may be derived from various species, including, human, cynomolgus monkey, rat, mice, camel, llama, shark, rabbit, and the like. In one embodiment, the antibody is derived from a human or cynomolgus monkey. In one embodiment, the antibody is derived from a human. In one embodiment, when the antibody is derived from a non-human species, to the antibody is chimerized or humanized using well-known techniques. In one embodiment, the antibody may be a polyclonal antibody or a monoclonal antibody. In one embodiment, the antibody is a monoclonal antibody.

In one embodiment, the polypeptide according to the present disclosure may be a “functional fragment of an antibody,” in which the fragment includes a variant Fc region comprising the above-mentioned specific amino acid substitutions. A ““functional fragment of an antibody,” also referred to as an “antigen-binding fragment of an antibody,” means a partial fragment of an antibody having antigen-binding activity, and includes, but is not limited to, a linear antibody fragment (see, for example, U.S. Pat. No. 5,641,870) and a multispecific antibody antibody fragment. An antigen-binding fragment includes fragments generated by treating a full-length antibody molecule with an appropriate enzyme, as well as proteins produced in appropriate host cells using genetically engineered antibody genes (i.e., recombinant proteins). Functional fragments also include, for example, an antigen-binding fragment comprising asparagine (Asn297), which undergoes modification with a N-linked sugar chain well-conserved in the Fc region of an IgG heavy chain, as well as neighboring amino acids.

In one embodiment, the polypeptide may be a “bispecific antibody” or a “multispecific antibody.” A multispecific antibody may be an antibody comprising a plurality of antigen-binding domains, where each antigen-binding domain binds to a different antigen. A bispecific antibody is an antibody that binds comprises two antigen-binding domains, where each antigen-binding domain binds to a different antigen. In both of these cases, amino acid substitutions may be included in one or both of the two CH2 domains in the antibody Fc region, and when included in both, the same or different amino acid substitutions may be included. Alternatively, a multispecific antibody may be an artificial protein where a plurality of antibodies each having a different antigen-binding domain are bound to each other, and a bispecific antibody may be an artificial protein in which two antibodies having a different antigen-binding domains are bound to each other. In these cases, amino acid substitutions may be included in one or both of the two CH2 domains in the Fc region of at least one antibody of the plurality of antibodies, and when included in both, the same or different amino acid substitutions may be included. Introduction of different amino acid substitutions into the two CH2 domains can be carried out by using a technique that allows production of antibody molecules from two different heavy chains (a heterodimer), such as the “knobs-into-holes” technique (International Publication No. WO 1998/050431), “electrostatic steering” (J Biol Chem, 285:19637-46 (2010)), strand exchange engineered domain (SEED) technology (Prot Eng Des Sel, 23(4):195-202 (2010)), or the technique described in International Publication Nos. WO 2012/058768 and WO 2013/063702. The bispecific or multispecific antibody may also be generated as a full-length antibody or fragment thereof (see, for example, International Publication No. WO 96/16673, and U.S. Pat. No. 5,837,234). Method for producing bispecific or multispecific antibodies are well known in the art and include, for example, a method for co-expressing two immunoglobulin heavy chain-light chain pairs, where the two chains have different specificities (see, for example, Millstein et al., Nature, 305:537-539 (1983)); techniques for producing bispecific antibodies from antibody fragments (see Brennan et al., Science, 229:81 (1985)), as well as other methods known in the art.

In one embodiment, the antibody may be a “chimeric antibody.” A “chimeric antibody” means an antibody comprising one or more regions derived from one antibody and one or more regions derived from one or more other antibodies. For example, an Fc sequence may be derived from a human antibody and a variable region sequence may be derived from a non-human species antibody such as a cynomolgus monkey.

In one embodiment, the antibody may be a “human antibody.” A “human antibody” includes all antibodies having one or more variable regions and constant regions derived from a human immunoglobulin sequence. In one embodiment, all variable and constant domains of an antibody are derived from a human immunoglobulin sequence (referred to as a “fully human antibody”). These antibodies can be prepared in a variety of ways, such as immunization of a non-human animal genetically modified to express antibodies derived from genes encoding human heavy and/or light chains.

In one embodiment, the antibody may be a “humanized antibody.” A “humanized antibody” is an antibody produced by inserting an antigen-binding site (such as one or more CDRs) of an antibody produced in a non-human species into a sequence of a human antibody. Humanized antibodies include human immunoglobulins in which residues from a hypervariable region are replaced by residues from a hypervariable region of a non-human animal having a desired specificity, affinity, etc., or in which an Fv framework region (FR) residue of a human immunoglobulin is replaced by a corresponding non-human residue. Humanized antibodies, when administered to a human subject, are less likely to elicit an immune response and/or induce a less severe immune response compared to antibodies from a non-human species.

In one embodiment, the antibody may be a “single-chain antibody.” A “single-chain antibody” refers to an antibody in which the heavy chains, which are originally double-stranded, are connected by a linker to form a single chain (see, for example, Biomaterials, 117:24-31 (2017)). Thus, single chain antibodies also include two CH domains, and the specific amino acid substitutions described above may be introduced into one or both of the CH2 domains.

In general, antibodies can be obtained by immunizing an animal with a polypeptide serving as an antigen using methods commonly practiced in the field and collecting and purifying the antibody produced in vivo. The origin of the antigen is not limited to humans, and an antigen derived from a non-human animal, such as a cynomolgus monkey, a mouse, a rat, can also be used to immunize an animal to generate antibodies. The antigen may one of a variety of antigens, for example, a cytokine, a soluble or insoluble factor, a molecule expressed on a pathogen, a molecule expressed on a cell, or a molecule expressed on a cancer cell. In some embodiments, the antigen is a cancer antigen. A monoclonal antibody can be obtained, for example, by establishing hybridomas by fusing myeloma cells with antibody-producing cells using known methods (see, for example, Kohler and Milstein, Nature, 256:495-497 (1975), Kennett, R. ed., Monoclonal Antibodies, p. 365-367, Plenum Press, N.Y. (1980)). Other methods are known in the art. Antibodies applicable to human diseases can also be selected by testing cross-over of human antigens with antibodies that bind to obtained heterologous antigens.

In the antibodies produced in cultured mammalian cells, it is known that the lysine residue at the carboxyl end of the heavy chain is deleted (Journal of Chromatography A, 705:129-134 (1995)), two amino acid residues of glycine and lysine at the heavy chain carboxyl end are also deleted, and the proline residue located at the carboxyl end is newly amidated (Analytical Biochemistry, 360:75-83 (2007)). However, deletion or modification of these heavy chain sequences does not affect an ability of the antibody to bind to an antigen or the effector functions of the antibody (such as complement activation and antibody-dependent cytotoxicity). Thus, antibodies according to the present disclosure also include antibodies that have undergone such deletion or modification and functional fragments of such antibodies, including an antibody having deletions in which one or two amino acids are deleted at the heavy chain carboxyl terminus, and an amidated antibody having deletions (for example, a heavy chain in which a proline residue at the carboxyl terminal site is amidated, and a heavy chain lacking lysine residue at the carboxyl terminus) and the like. However, as long as the antigen-binding ability and the reduced effector function are maintained, deletions at the carboxyl terminal of the heavy chain of the antibody is not limited to the above examples. The two heavy chains constituting the antibody may be those derived from any one of the heavy chain from a full length antibody, an antibody having deletions as described above, or a combination of both. Although the ratio of each deletion may be affected by the type of cultured mammalian cells producing the antibody and the culture conditions, in one embodiment the antibody is one in which one amino acid residue at the carboxyl terminus is deleted in both of the two heavy chains.

in one embodiment, when the polypeptide according to the present disclosure is an antibody, the antibody is an antibody that targets tumor cells or immune cells. In one embodiment, the antibody is an antibody that targets tumor cells. In one embodiment, the polypeptide is an antibody or functional fragment thereof that binds a tumor antigen. In one embodiment, when the polypeptide is an antibody targeting a tumor cell, the antibody has one or more of the following characteristics: is capable of recognizing the tumor cell, is capable of binding to the tumor cell, is capable of being incorporated into and internalized into the tumor cell, and/or damages the tumor cell.

In one embodiment, when the polypeptide according to the present disclosure is an antibody targeting tumor cells, the antibody may be, for example, an anti-HER2 antibody, an anti-HER3 antibody, an anti-DLL3 antibody, an anti-FAP antibody, an anti-CDH11 antibody, an anti-CDH6 antibody, an anti-A33 antibody, an anti-CanAg antibody, an anti-CD19 antibody, an anti-CD20 antibody, an anti-CD22 antibody, an anti-CD30 antibody, an anti-CD33 antibody, an anti-CD56 antibody, an anti-CD70 antibody, an anti-CD98 antibody, an anti-LPS antibody, an anti-TROP2 antibody, an anti-CEA antibody, an anti-Cripto antibody, an anti-EphA2 antibody, an anti-G250 antibody, an anti-MUC1 antibody, an anti-GPNMB antibody, an anti-Integrin antibody, an anti-PSMA antibody, an anti-Tenascin-C antibody, an anti-SLC44A4 antibody, an anti-mesothelin antibody, an anti-ENPP3 antibody, an anti-CD47 antibody, an anti-EGFR antibody or an anti-DR5 antibody. In one embodiment, when the polypeptide according to the present disclosure is an antibody targeting tumor cells, the antibody may be an anti-CD70 antibody, an anti-LPS antibody or an anti-EGFR antibody. Such antibodies have been prepared by known methods (see, for example, Proc. Natl. Acad. Sci. USA, 81:6851-6855 (1984); Nature 3211:522-525 (1986), and International Publication No. WO 90/07861). See also for anti-CD70 antibody (International Publication Nos. WO 2004/073656 and WO 2007/038637), for anti-LPS antibody (International Publication Nos. WO 2015/046505 and WO 2019/065964), and for anti-EGFR antibody (International Publication Nos. WO 1998/050433 and WO 2002/092771).

In one embodiment, when the polypeptide according to the present disclosure is an antibody targeting tumor cells, the antibody may be panitumumab, alemtuzumab, atezolizumab, avelumab, belimumab, blinatumomab, glenbatumumab, ibritumomab, ipilimumab, nivolumab, brentuximab, canakinumab, cetuximab, daratumumab, denosumab, pidilizumab, mogamulizumab, ramucirumab, rituximab, siltuximab, dinutuximab, durvalumab, elotuzumab, obinutuzumab, ofatumumab, olaratumab, pembrolizumab, pertuzumab, tavolixizumab, tositumomab, trastuzumab, tremelimumab, varlilumab, vorsetuzumab, or O11-1111. In one embodiment, when the polypeptide according to the present disclosure is an antibody targeting tumor cells, the antibody may be panitumumab, vorsetuzumab, or O11-1111.

In one embodiment, the polypeptide according to the present disclosure can be an antibody that targets tumor cells and further may be used for the treatment of, for example, inflammatory diseases, immune diseases, infectious diseases and the like, or regenerative medicine, and the like.

In addition, in some embodiments, the polypeptide according to the present disclosure may be, for example, a monovalent antibody, a strand exchange engineered domain (SEED), a triomab, a double variable domain immunoglobulin (DVD-Ig), a miniantibody, a double-affinity re-targeting molecule (Fc-DART or Ig-DART), a LUZ-Y antibody, a Biclonic antibody, a double-targeting (DT)-Ig antibody, a two-in-one antibody, a crosslinked Mab, a mAb², a CovX-body, a Ts2Ab, a BsAb, a HERCULES antibody, a TvAb, or a SCORPION.

### Fusion Proteins

Certain embodiments of the present disclosure relate to a “fusion protein” comprising the variant Fc region or a polypeptide or an antibody moiety comprising the variant Fc region fused to another protein or polypeptide (a “heterologous” protein or polypeptide). Various heterologous proteins or polypeptides can be fused to the variant Fc region, polypeptide or antibody moiety to produce the fusion protein using recombinant techniques. Examples include, but are not limited to, receptors or target-binding regions thereof, adhesion molecules, ligands, enzymes, cytokines, immunocytokines, non-immunoglobulin proteins, chemokines, various protein domains, and the like. Also, other proteins of interest include therapeutic agents that direct the fusion protein to a therapeutic target, and such targets may be a molecule associated with a disease, such as a receptor protein that binds to a target. Examples of fusion proteins comprising a receptor protein that binds to a target include, but are not limited to, VEGFR-Fc fusions, TNFR-Fc fusions, CTLA4-Fc fusions, and the like. Fusion proteins may also comprise an scFv, which may be constructed by fusing the Fc region either at the amino- or carboxy-terminus of the scFv (see, for example, Antibody Engineering, ed. Borrebaeck, 1995, Oxford University Press). It is understood that a “fusion protein” is one embodiment of the above-mentioned “molecule comprising the polypeptide according to the present disclosure.”

### Polypeptide-Drug Conjugates

Certain embodiments of the present disclosure relate to polypeptide-drug conjugates in which the polypeptide according to the present disclosure is conjugated to one or more drugs or modifying agents. In one embodiment, the polypeptide-drug conjugates according to the present disclosure encompass modified antibodies. For example, an antibody may be conjugated to a chemotherapeutic agent, a cytotoxic agent, or a variety of non-proteinaceous polymers, such as polyethylene glycol, polypropylene glycol, polyoxyalkylene, or a copolymer of polyethylene glycol and polypropylene glycol. A polypeptide-drug conjugate in which the polypeptide is conjugated to both a drug and a polymer (modifying agent) is also encompassed in certain embodiments.

A polypeptide-drug conjugate may be prepared by conjugating the polypeptide to the drug or modifying agent via a linker. Linkers typically include a functional group capable of reacting with the target group or groups on the antibody and one or more functional groups capable of reacting with a target group on the drug or modifying agent. Suitable functional groups and linkers are known in the art and include those described, for example, in Bioconjugate Techniques (G. T. Hermanson, 2013, Academic Press) and Antibody-Drug Conjugates: Methods in Molecular Biology (Ducry (Ed.), 2013, Springer).

### Pharmaceutical Compositions

In certain embodiments the polypeptide, the molecule comprising said polypeptide, or the polypeptide-drug conjugate may be provided in a pharmaceutical composition comprising a pharmaceutically acceptable carrier. Pharmaceutically acceptable carriers include excipients, inert diluents, granulating agents, disintegrating agents, binders, wetting agents, colorants, preservatives, aqueous vehicles and solvents, oily vehicles and solvents, surfactants, dispersing agents, sweeteners, perfumes, emulsifiers, lubricants, buffers, thickening agents, fillers, antioxidants, stabilizers, and the like. Suitable pharmaceutically acceptable carriers are known in the art and are described, for example, in “Remington: The Science and Practice of Pharmacy” (formerly “Remington's Pharmaceutical Sciences”” by E. W. Martin), Lippincott, Williams & Wilkins, Philadelphia, PA (2000).

In one embodiment, the pharmaceutical composition described above may also include one or more other “therapeutic agents,” or may be used or administered in combination with one or more other “therapeutic agents”. For example, when used for cancer treatment purposes, examples of other therapeutic agents for cancer treatment include, but are not limited to, abraxane, carboplatin, cisplatin, gemcitabine, irinotecan (CPT-11), paclitaxel, pemetrexed, sorafenib, vinblastine or drugs described in International Publication No. WO 2003/038043, as well as LH-RH analogs (leuprorelin, goserelin, etc.), estramustine phosphates, estrogen antagonists (tamoxifen, raloxifene, etc.), aromatase inhibitors (anastrozole, letrozole, exemestane, etc.), immunocheckpoint inhibitors (nivolumab, ipilimumab, etc.), and the like. The route of administration of the pharmaceutical composition includes, but is not limited to, an administration via intradermal, intramuscular, intraperitoneal, intravenous, or subcutaneous routes. When combined with other therapeutic agents, the pharmaceutical composition and the one or more other therapeutic agents can be used or administered simultaneously or at different times, either as a single composition or separately.

### Methods of Use

In certain embodiments, the polypeptide, the molecule comprising the same, or the polypeptide-drug conjugate can be used in a method for the treatment of humans. For example, in one embodiment, the polypeptide, the molecule comprising the same, or the polypeptide-drug conjugate can be used in the treatment of a cancer or tumor, an immune disease, an inflammatory disease or an infectious disease. In one embodiment, the polypeptide, the molecule comprising the same, or the polypeptide-drug conjugate can be used in a method of diagnosis in humans. In one embodiment, the polypeptide, the molecule comprising the same, or the polypeptide-drug conjugate can be used for the treatment of tumors or cancer in humans. Tumor diseases and cancers include, but are not limited to, lung cancer, renal cancer, urothelial cancer, colorectal cancer, prostate cancer, glioblastoma multiforme, ovarian cancer, pancreatic cancer, breast cancer, melanoma, liver cancer, bladder cancer, gastric cancer, esophageal cancer, uterine body cancer, testicular cancer, cervical cancer, placental choriocarcinoma, glioblastoma multiforme, brain tumor, head/neck cancer, thyroid cancer, mesothelioma, gastrointestinal stromal tumor (GIST), gallbladder cancer, bile duct cancer, adrenal carcinoma, squamous cell carcinoma, leukemia, malignant lymphoma, myeloma or sarcoma, etc.

### Additional Amino Acid Modifications

In addition to the specific amino acid substitution(s) described above (for example, LALA-DG, LALA-DGPA, and LALA-DN mutations), the polypeptides may comprise additional amino acid modification(s) in the Fc region. Thus, similarly, the “variant Fc region” of the polypeptide according to the present disclosure may also comprise additional amino acid modification(s) in the Fc region in addition to the specific amino acid substitutions described above (for example, LALA-DG, LALA-DGPA, and LALA-DN). The additional amino acid modifications can be made not only to the Fc region of the polypeptide but also to one or more other regions of the polypeptide.

An “amino acid modification” refers to a substitution, deletion, addition, insertion, modification, etc., of an amino acid, and can be made by various methods known in the art. For example, without limitation, amino acid substitutions, additions, deletions, and insertions, can be made using any well-known PCR-based technique, and amino acid substitutions can be made by site-directed mutagenesis (see, for example, Zoller and Smith, Nucl. Acids Res 10:6487-6500 (1982); Kunkel, Proc. Natl. Acad. Sci USA, 82:488 (1985)). Examples of “modification” of an amino acid includes addition or deletion of a sugar chain.

In one embodiment, the amino acid substitution(s) may be “conservative amino acid substitution(s).” A “conservative amino acid substitution” means that an amino acid residue is substituted by another amino acid residue having a side chain group having similar chemical properties (for example, charge or hydrophobicity) and generally does not substantially alter the functional properties of the protein. Examples of conservative amino acid substitutions are well known in the art and may be made, for example, by making substitution(s) within a class of amino acids represented in one or more of the following: acidic residues: Asp, Glu; basic residues: Lys, Arg, His; hydrophilic uncharged residues: Ser, Thr, Asn, Gln; aliphatic uncharged residues: Gly, Ala, Val, Leu, Ile; nonpolar uncharged residues: Cys, Met, Pro, and aromatic residues: Phe, Tyr, Trp.

The additional amino acid modification(s) may be at least one amino acid modification, for example, about 20 or fewer, about 10 or fewer, or about 1 to about 5 amino acid modifications. When the additional amino acid modification(s) is/are made to a wild-type sequence (for example, the sequence of a wild-type Fc region), it is preferred that the sequence of the variant sequence (for example, the variant Fc region) has at least about 80%, at least about 90%, at least about 95%, at least about 96%, at least about 97%, at least about 98%, at least about 99% homology to the corresponding wild-type sequence. In one embodiment, the sequence of the variant sequence (for example, the variant Fc region) has at least about 80%, at least about 90%, at least about 95%, at least about 96%, at least about 97%, at least about 98%, at least about 99% identity to the corresponding wild-type sequence. The sequence similarity or identity of polypeptides can be measured using known sequence analysis software (for example, GAP, BESTFIT, FASTA or TFASTA in the Wisconsin Genetics Software Package, Genetics Computer Group, Madison, WI).

Examples of additional amino acid modifications include amino acid substitutions, such as G236A, G236P, G236L, G236K, G236R, G236T, G236H, G236N, G237R, P238I, S239M, S239K, S239R, S239T, S239H, S239H, S239Q, V266I, V266L, S267P, S267K, H268A, H268V, H268F, H268P, H268M, H268I, H268L, H268K, H268R, H268T, H268Y, H268Q, H268W, E269A, E269V, E269D, E269K, E269R, E269S, E269T and E269H (all positions expressed by EU numbering), which have been shown to increase the Tm of an antibody Fc over the parent polypeptide, as described in International Publication No. WO 2013/118858.

In one embodiment, an amino acid substitution which further reduces Fcγ receptor binding activity may be used. Examples of additional amino acid substitutions that reduce binding activity to an Fcγ receptor, include, for example, amino acid substitutions such as G237F/S239E/A327H, G237/A327/A330I, S239E/S267E/H268D (all positions expressed by EU numbering), as shown in International Publication No. WO 2011/120134.

In one embodiment, the additional amino acid modifications include substitutions that reduce sensitivity to proteolysis, substitutions that reduce sensitivity to oxidation, substitutions that alter binding affinity to form protein complexes, substitutions that impart or modify other physicochemical or functional properties of such analogs, modifications that suppress a deamidation reaction, and the like.

### Methods of Preparation

Polypeptides of the present disclosure may be prepared by recombinant protein methods known in the art. Without limiting to the following method, a polypeptide according to the present disclosure may be obtained by a method comprising the following steps: first designing a DNA encoding a polypeptide comprising the variant Fc region described above, then chemically synthesizing the DNA and inserting the DNA into a vector, or the like, followed by introducing the DNA/vector into a host cell, culturing the host cell in a medium under conditions suitable for expression of the polypeptide, and recovering the polypeptide from the medium or from the cells. A vector comprising a DNA which encodes a polypeptide comprising a variant Fc region can be prepared, for example, by introducing relevant mutations into a polypeptide comprising a wildtype Fc region, for example, using KOD-Plus-Mutagenesis kit (Toyobo Co., Ltd., Osaka, Japan) according to the manufacturer's instructions. When the polypeptide comprises other amino acid modifications (for example, additions, deletions, substitutions, etc.) of interest other than those described above, a DNA sequence encoding a polypeptide comprising the specific amino acid substitutions for the variant Fc region described above and the other amino acid modification(s) of interest is designed and the polypeptide can be obtained by the same method as described above. In addition, the polypeptide of the present disclosure may also be prepared by peptide synthesis, chemical synthesis, in vitro translation, and the like, in addition to recombinant methods.

Thus, in one embodiment, the present disclosure relates to a nucleic acid comprising a nucleotide sequence encoding a polypeptide comprising the variant Fc region described above or a portion thereof, such as a heavy chain or a light chain. In one embodiment, the present disclosure relates to a vector comprising said nucleic acid. In one embodiment, the present disclosure relates to a host cell comprising a nucleic acid encoding a polypeptide comprising the variant Fc region described above. In one embodiment, the present disclosure relates to a method for producing a polypeptide described herein, for example, comprising a step of culturing host cells comprising a nucleic acid encoding said variant Fc region or a polypeptide comprising the same in a medium under conditions suitable for expression of the variant Fc region or polypeptide comprising the variant Fc region, and recovering the variant Fc region or polypeptide from the medium or the cells.

### Embodiments

Exemplary non-limiting embodiments of the present disclosure include the following:

[1] A polypeptide comprising one or more variant IgG1 Fc regions or variant IgG4 Fc regions, wherein each of the variant Fc regions comprises two CH2 domains, wherein one or both of the CH2 domains in at least one of the variant Fc regions is/are variant CH2 domain(s), and wherein amino acid residues at positions 234, 235, and 265 (all positions expressed by EU numbering) of the variant CH2 domain are Ala, Ala, and Gly, respectively.

[2] The polypeptide according to [1], wherein the amino acid residue at position 329 of the variant CH2 domain is Ala.

[3] A polypeptide comprising one or more variant IgG1 Fc regions or variant IgG4 Fc regions, wherein each of the variant Fc regions comprises two CH2 domains, wherein one or both of the CH2 domains in at least one of the variant Fc regions is/are variant CH2 domain(s), and wherein amino acid residues at positions 234, 235, and 265 (all positions expressed by EU numbering) of the variant CH2 domain are Ala, Ala, and Asn, respectively.

[4] The polypeptide according to [1] or [2], wherein a thermal denaturation midpoint temperature (Tm) of the variant CH2 domain is higher than that of a parent CH2 prior to introducing mutations by 1° C. or more.

[5] The polypeptide according to [4], wherein the thermal denaturation midpoint temperature (Tm) of the variant CH2 domain is higher than that of the parent CH2 by 3° C. or more.

[6] The polypeptide according to [3], wherein a thermal denaturation midpoint temperature (Tm) of the variant CH2 domain is not higher than that of a parent CH2 prior to introducing mutations by 1° C. and is not lower than that of the parent CH2 prior to introducing mutation by 1° C.

[7] The polypeptide according to any one of [1] to [6], wherein the polypeptide has reduced binding affinity for at least one Fcγ receptor as compared to a parent polypeptide comprising a parent CH2 domain prior to introducing mutations.

[8] The polypeptide according to [7], wherein the polypeptide has reduced binding affinity for at least one Fcγ receptor by 50% or more as compared to the parent polypeptide.

[9] The polypeptide according to [7] or [8], wherein said at least one Fcγ receptor is at least one selected from a human Fcγ receptor and a cynomolgus Fcγ receptor.

[10] The polypeptide according to [9], wherein the Fcγ receptor is a human Fcγ receptor.

[11] The polypeptide according to any one of [7] to [10], wherein the Fcγ receptor is FcγRI.

[12] The polypeptide according to any one of [1] to [11], wherein the polypeptide comprises one or more variant IgG1 Fc regions.

[13] The polypeptide according to any one of [1] to [12], wherein the polypeptide is an antibody or a functional fragment thereof.

[14] The polypeptide according to [13], wherein the antibody or functional fragment thereof binds a tumor antigen.

[15] A molecule comprising the polypeptide according to any one of [1] to [14].

[16] The molecule according to [15], which the molecule is a fusion protein of the polypeptide according to any one of [1] to [14] and a polypeptide that is not said polypeptide.

[17] A polypeptide-drug conjugate in which the polypeptide according to any one of [1] to [14] is conjugated to a drug.

[18] A nucleic acid comprising a nucleotide sequence encoding the polypeptide according to any one of [1] to [14] or a portion thereof.

[19] A host cell comprising the nucleic acid according to [18].

[20] A pharmaceutical composition comprising the polypeptide according to any one of [1] to [14], the molecule according to [15] or [16], or the polypeptide-drug conjugate according to [17], and a pharmaceutically acceptable carrier.

[21] A polypeptide according to any one of [1] to [14], the molecule according to [15] or [16], or polypeptide-drug conjugate according to [17], for use in therapy.

[22] The polypeptide, molecule or polypeptide-drug conjugate for use according to [21], wherein the therapy comprises treatment of a cancer, an immune disease, an inflammatory disease or an infectious disease.

### EXAMPLES

The following Examples are provided for illustrative purposes and are not intended to limit the scope of the invention in any way.

### Comparative Example 1: Evaluation of Binding Activity of an Anti-EGFR Antibody with Known Effectorless Mutations to Human FcγRI

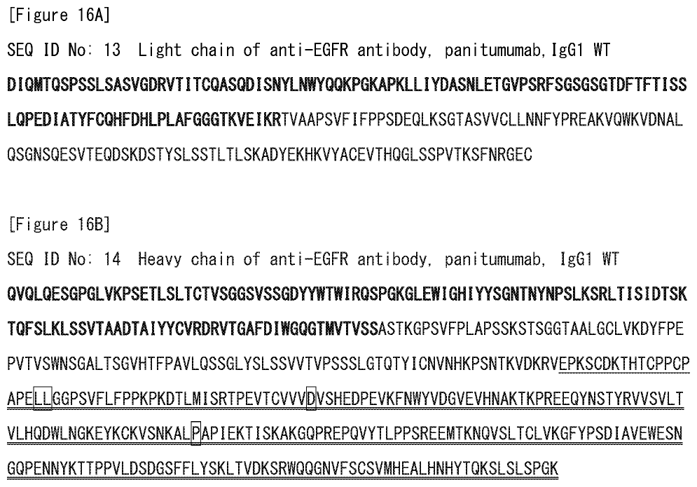

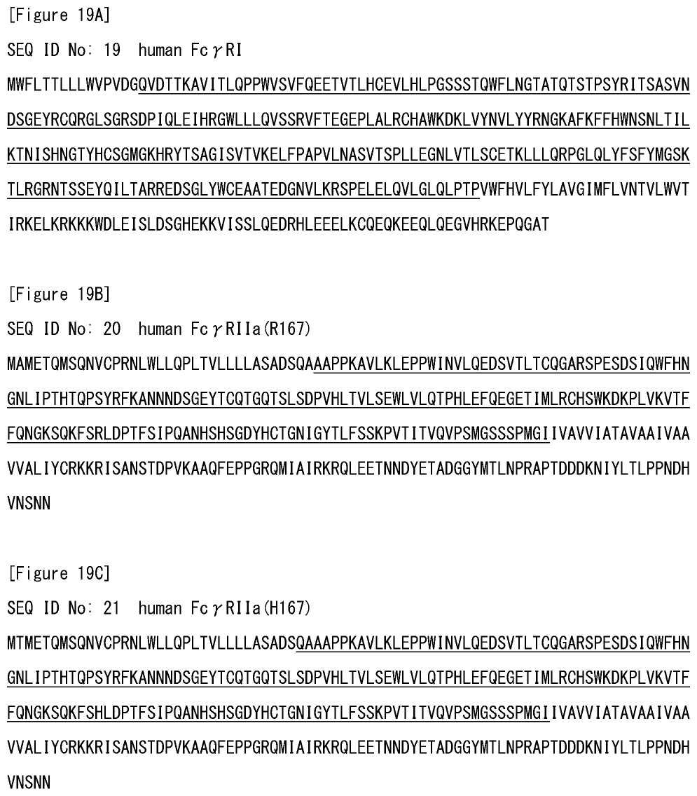

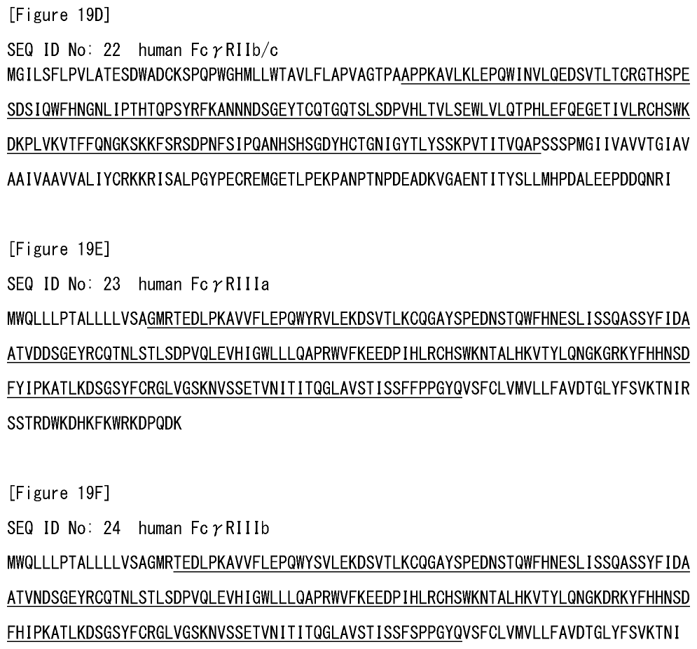

The binding activity of an anti-EGFR antibody, into which known effectorless mutations were introduced, to human FcγRI (hFcγRI), was evaluated using Biacore™ T200 (Cytiva, Marlborough, MA). The Fc region of anti-EGFR antibody Panitumumab, which is originally a human IgG2 antibody, was replaced by that of human IgG1 and the resulting antibody is termed to IgG1 WT. The amino acid sequences of the heavy chain and the light chain of IgG1 WT are represented by SEQ ID Nos: 13 and 14, respectively (see FIGS. 16A and 16B). IgG1 WT and the following variants were evaluated: a variant obtained by incorporating L234A/L235A (LALA) mutations into the Fc region of IgG1 WT; a variant obtained by introducing L234A/L235A/D265A (LALA-DA) mutations into the Fc region of IgG1 WT; and a variant obtained by introducing L234A/L235A/P329G (LALA-PG, described in International Publication No. WO 2012/130831) mutations into the Fc region of IgG1 WT. The noted mutations were introduced into both CH2 domains of the Fc region. These anti-EGFR antibodies (also referred as “variants of the anti-EGFR antibody”) were obtained by: introducing a vector comprising a DNA encoding the amino acid sequence of the antibody into host cells; culturing the host cells in medium thereby letting the host cells express the DNA in said cells. A His-tagged human FcγRI consisting of recombinant human Fc gamma RI/CD64 Protein, CF (R&D Systems, Minneapolis, MN, Accession #: P12314) comprising the Gln residue at position 16 to the Pro residue at position 288 of SEQ ID No: 19 (see FIG. 19A), to which 6×His tag was added at the carboxyl terminus, was used as a ligand. The FcγRI ligand was captured on a sensor chip on which anti-His antibodies were immobilized. Various anti-EGFR antibodies as analytes were then added to the sensor chip to measure an interaction between the ligand and the analyte by surface plasmon resonance (SPR) method.

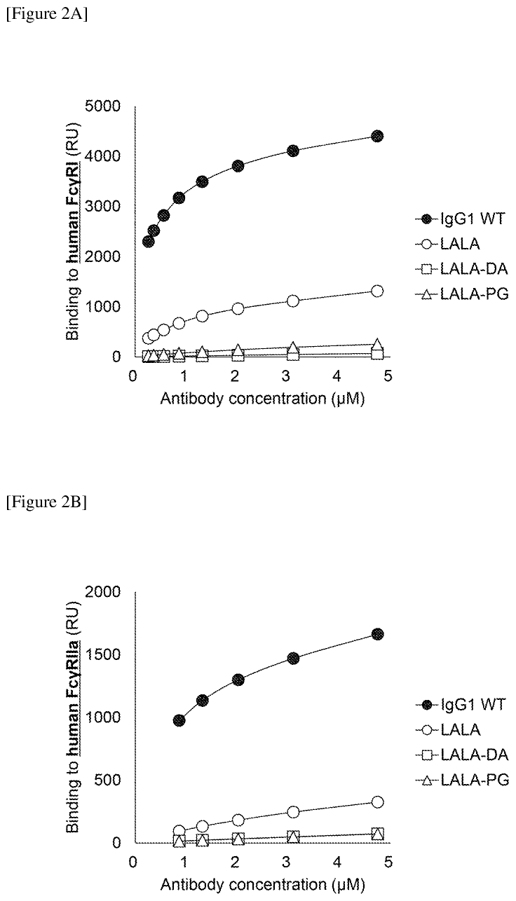

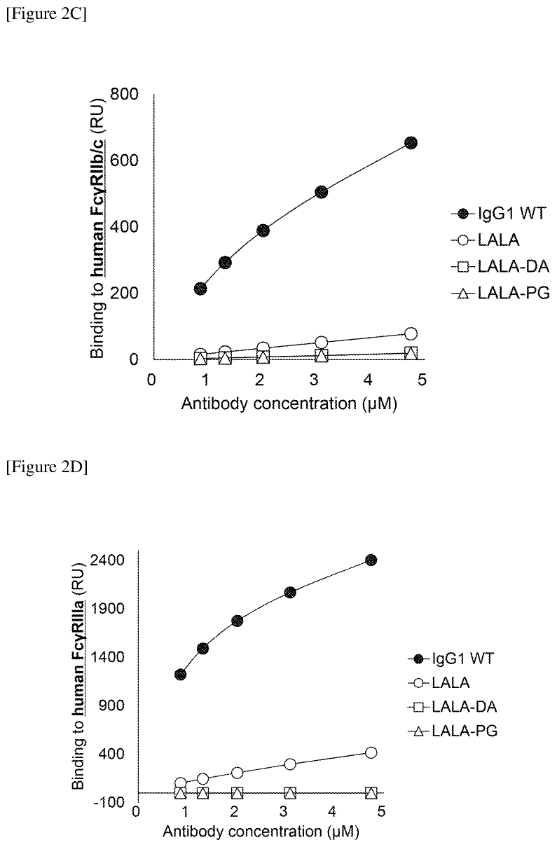

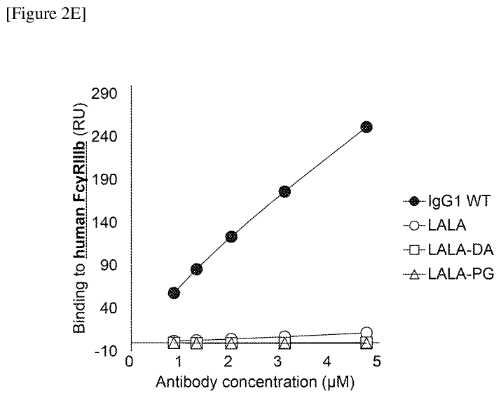

Briefly, anti-6×His tag antibodies [AD1.1.10] (Abcam plc, Cambridge, UK) were immobilized on Sensor Chip CM5 (Cytiva, Marlborough, MA) according to the manufacturer's instructions, and then Recombinant Human Fc gamma RI/CD64 Protein (R & D Systems) prepared at 10 μg/mL in HBS-EP+ (GE Healthcare, Chicago, IL) was captured by contacting it with the sensor chip at 10 μL/min for 60 seconds. Then, the anti-EGFR antibody prepared at each of the given concentrations in HBS-EP+ was added to the sensor chip at a flow rate of 30 μL/min for 120 seconds. FIG. 2A shows a graph of binding response (Resonance Unit, RU) plotted against each of the tested antibody concentrations (0.3, 0.4, 0.6, 0.9, 1.3, 2.0, 3.1, and 4.7 μM). Table 1 below shows the RU values obtained at an antibody concentration of 4.7 μM.

TABLE 1
Binding Activity of Variants and IgG1 WT
to Various Human Fcγ Receptors

| | | | hFcγRIIa | |
|---|---|---|---|---|
| | hFcγRI | hFcγRIIa | (H167) | hFcγRIIb/c |
| IgG1 WT | 4.4 × 10³ | 1.7 × 10³ | 1.4 × 10³ | 6.5 × 10² |
| LALA | 1.3 × 10³ | 3.3 × 10² | 1.6 × 10² | 7.8 × 10 |
| LALA-DA | 7.0 × 10 | 7.3 × 10 | 2.4 × 10 | 2.0 × 10 |
| LALA-PG | 2.5 × 10² | 7.2 × 10 | 1.9 × 10 | 1.9 × 10 |

| | | hFcγRIIIa | |
|---|---|---|---|
| | hFcγRIIIa | (V176F) | hFcγRIIIb |
| IgG1 WT | 2.4 × 10³ | 5.3 × 10² | 2.5 × 10² |
| LALA | 4.2 × 10² | 4.5 × 10 | 1.2 × 10 |
| LALA-DA | 0.4 | −2.2 | 0.3 |
| LALA-PG | −0.7 | −0.8 | −0.4 |

As shown in Table 1 above, all variants with the effectorless mutations had a lower RU value, which is an indication of the binding to human FcγRI, than IgG1 WT. The RU values of the variants were in the order of LALA>LALA-PG>LALA-DA.

### Comparative Example 2: Evaluation of Binding Activity of Anti-EGFR Antibodies with Known Effectorless Mutations to Various Human FcγR

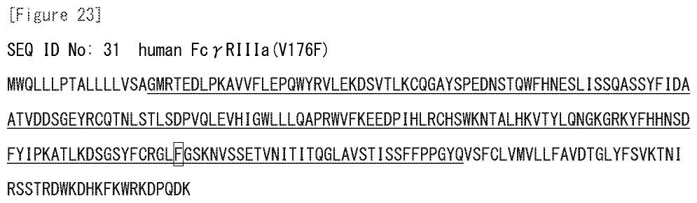

Biacore™ T200 was used to evaluate the binding activity of the anti-EGFR antibodies described in Comparative Example 1 to human FcγRIIa, FcγRIIa (H167), FcγRIIb/c, FcγRIIIa, FcγRIIIa (V176F), and FcγRIIIb. After immobilizing human FcγR as a ligand directly on the sensor chip, various anti-EGFR antibodies as analysts were added to the sensor chip to measure an interaction between the ligand and the analysts by the SPR method. The following FcγRs were used, each comprising either a 10×His tag or a 6×His tag at the carboxyl terminus, and all obtained from R & D Systems (Minneapolis, MN): Recombinant Human Fc gamma RIIA/CD32a (R167) Protein (Accession #: AAA35827, comprising the Ala residue at position 36 to the Ile residue at position 218 of SEQ ID No: 20 (see FIG. 19B), with a 10×His tag), Recombinant Human Fc gamma RIIA/CD32a (H167) Protein (Accession #: P12318-1 (P12318.4), comprising the Ala residue at position 34 to the Ile residue at position 218 of SEQ ID No: 21 (see FIG. 19C) with a 10×His tag), Recombinant Human Fc gamma RIIB/C (CD32b/c) Protein (Accession #: P31994, comprising the Ala residue at position 46 to the Pro residue at position 217 of SEQ ID No: 22 (see FIG. 19D) with a 10×His tag), Recombinant Human Fc gamma RIIIA/CD16a protein (Accession #: AAH17865, comprising the Gly residue at position 17 to the Gln residue at position 208 of SEQ ID No: 23 (see FIG. 19E) with a 6×His tag), Recombinant Human Fc gamma RIIIA/CD16a (V176F) Protein (Accession #: P08637, comprising the Gly residue at position 17 to the Gln residue at position 208 of SEQ ID No: 31 (see FIG. 23) with a 6×His tag), and Recombinant Human Fc gamma RIIIB/CD16b Protein (Accession #: O75015, comprising the Thr residue at position 20 to the Gln residue at position 208 of SEQ ID No: 24 (see FIG. 19F) with a 10×His tag).

Briefly, each of the human FcγRs was immobilized on the Sensor Chip CM5 according to the manufacturer's instructions. Subsequently, the anti-EGFR antibody prepared at each of the given concentrations in HBS-EP+ was added to the sensor chip at a flow rate of 30 μL/min for 120 seconds. FIGS. 2B to 2E show graphs in which RU values are plotted against each of the antibody concentrations (0.9, 1.3, 2.0, 3.1, and 4.7 μM) when human FcγRIIa, FcγRIIb/c, FcγRIIIa, and FcγRIIb were used as ligands. Table 1 shows the RU values for various human FcγRs obtained at an antibody concentration of 4.7 μM.

As can be seen from Table 1, all variants with effectorless mutations had lower RU values, an indication of binding to the respective human FcγR, than IgG1 WT. The RU values of the variants were in the order of LALA>LALA-PG, with LALA-DA being substantially the same as LALA-PG.

### Comparative Example 3: Evaluation of Binding Activity of Anti-EGFR Antibodies with Known Effectorless Mutations to Cynomolgus FcγRI

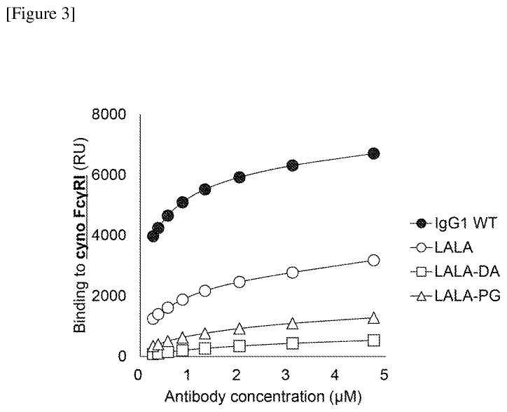

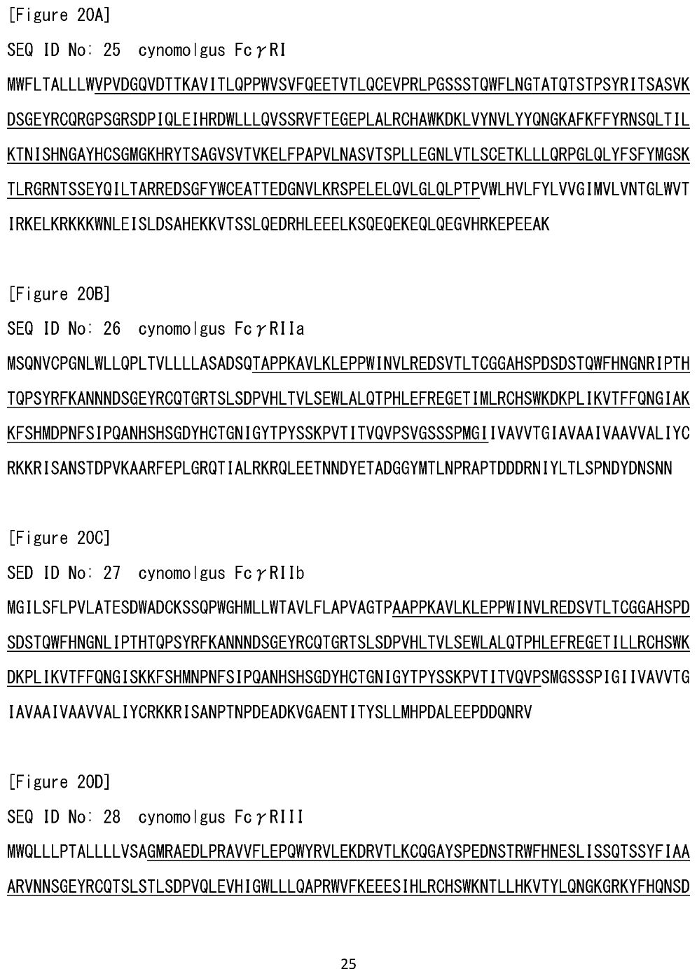

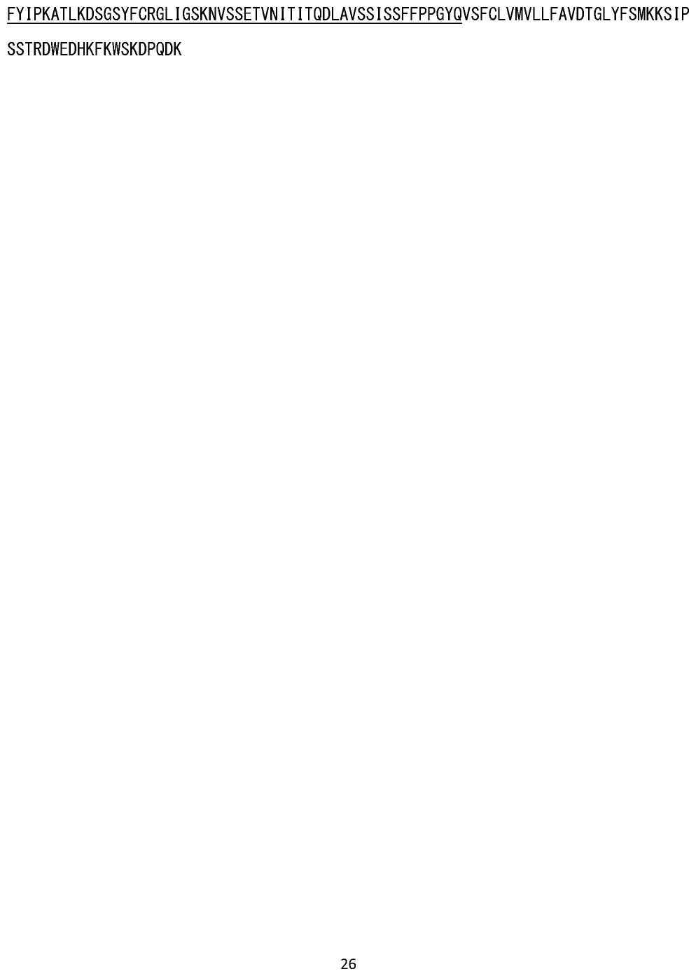

The binding activity of the anti-EGFR antibodies described in Comparative Example 1 to cynomolgus FcγRI (cFcγRI) were evaluated in the same manner as in Comparative Example 1. Cynomolgus Fc gamma RI/CD64 Protein (Accession #: NP_001270969, comprising the Val residue at position 11 to the Pro residue at position 288 of SEQ ID No: 25 (see FIG. 20A), to which a 6×His tag was added at the carboxyl terminus: R & D Systems, Minneapolis, MN) was used as the cynomolgus FcγR. FIG. 3A shows a graph in which the RU values are plotted against each of the concentrations of the antibodies (0.3, 0.4, 0.6, 0.9, 1.3, 2.0, 3.1, and 4.7 μM). Table 2 below shows the RU value obtained at an antibody concentration of 4.7 μM.

TABLE 2
Binding Activity of Variants and IgG1 WT
to Various Cynomolgus Fcγ Receptors

| | CFcγRI | cFcγRIIa | cFcγRIIb | CFcγRIII |
|---|---|---|---|---|
| IgG1 WT | 6.7 × 10³ | 9.1 × 10² | 8.4 × 10² | 2.8 × 10³ |
| LALA | 3.2 × 10³ | 9.4 × 10 | 2.7 × 10² | 5.4 × 10² |
| LALA-DA | 5.3 × 10² | 2.5 × 10 | 1.1 × 10² | 8.4 |
| LALA-PG | 1.3 × 10³ | 2.3 × 10 | 1.1 × 10² | 1.0 × 10 |

As shown in Table 2 above, all variants with the effectorless mutations had a lower RU value, indicative of binding to cynomolgus FcγRI, than IgG1 WT. The RU values of the variants were in the order of LALA>LALA-PG>LALA-DA.

### Comparative Example 4: Evaluation of Binding Activity of Anti-EGFR Antibodies with Known Effectorless Mutations to Various Cynomolgus FcγRs

The binding activity of the anti-EGFR antibodies described in Comparative Example 1 to cynomolgus FcγRIIa, FcγRIIb and FcγRIII was evaluated in the same manner as in Comparative Example 2. The following cynomolgus FcγRs were used, each comprising a 6×His tag at the carboxyl terminus, and all obtained from R & D Systems (Minneapolis, MN): Recombinant Cynomolgus Fc gamma RIIA/CD32a Protein (Accession #: NP_001270598, comprising the Thr residue at position 29 to the Ile residue at position 211 of SEQ ID No: 26 (see FIG. 20B), Recombinant Cynomolgus Fc gamma RIIB protein (Accession #: NP_001271060, comprising the Ala residue at position 46 to the Pro residue at position 217 of SEQ ID No: 27 (see FIG. 20C), and Recombinant Cynomolgus Fc gamma RIII/CD16 Protein (Accession #: NP_001270121, comprising the Gly residue at position 17 to the Gln residue at position 208 of SEQ ID No: 28 (see FIG. 20D) Table 2 above shows the RU values obtained at an antibody concentration of 4.7 μM.

As shown in Table 2 above, all variants with the effectorless mutations had a lower RU value, indicative of the binding to the respective cynomolgus FcγR, than IgG1 WT. The RU values of the variants were in the order of LALA>LALA-PG≥LALA-DA.

### Comparative Example 5: Evaluation of Thermal Stability of Anti-EGFR Antibodies with Known Effectorless Mutations

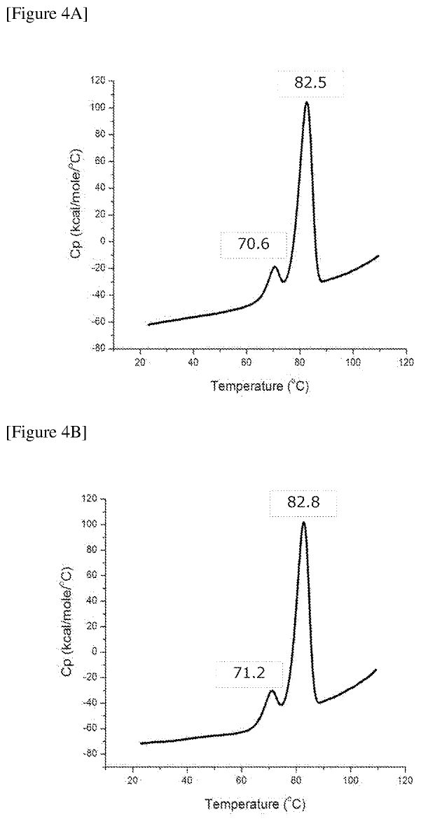

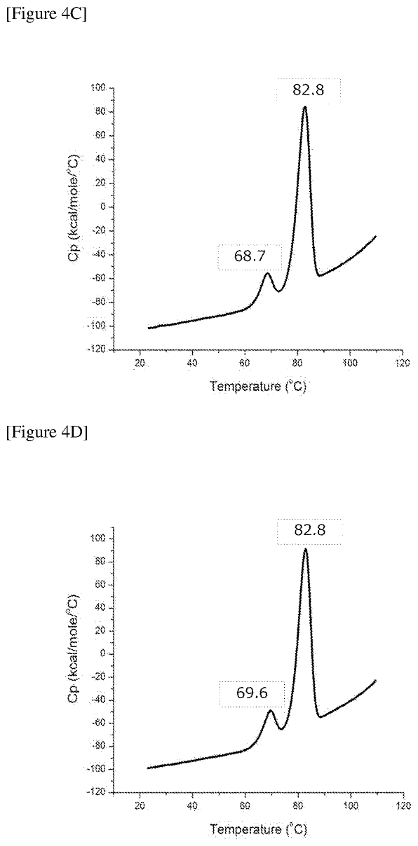

The thermal stability of the anti-EGFR antibodies described in Comparative Example 1 was evaluated using the MicroCal VP-Capillary DSC System (Malvern Panalytical Ltd., Malvern, UK). Each anti-EGFR antibody was prepared at an antibody concentration of 0.5 mg/mL in 25 mM histidine and 5% w/v sorbitol (pH 6.0), and changes in heat capacity Cₚ (kcal/mol/° C.) were examined when the temperature was raised from 20° C. to 120° C. at a heating rate of 60° C./hr. The peak of the curve showing the change in the heat capacity indicates the thermal denaturation of each of the domains of the antibody, and the peaks of the thermal denaturation are generally observed in the order of CH2 domain, Fab, and CH3 domain (Biochem. Biophys. Res. Commun. 355:751-757 (2007)). FIGS. 4A to 4D show the changes in the heat capacity and peak top temperature (Tₘ) for the IgG1 WT and LALA, LALA-DA, and LALA-PG variants thereof. Of the two peaks observed, the peak at the lower temperature indicates the thermal denaturation of the CH2 domain. The peak at the higher temperature is considered to indicate the overlapping peaks for the Fab region and the CH3 domain. Table 3 below shows the Tₘ value for the CH2 domain for each of the test antibodies and the difference in Tₘ values (ΔTₘ) between each variant and IgG1 WT.

TABLE 3
Tₘ of the CH2 Domain of Variants and IgG1 WT and
Difference in Tₘ (ATₘ) Between Variants and IgG1 WT

| | CH2 Tₘ (° C.) | ΔTₘ (° C.) |
|---|---|---|
| IgG1 WT | 70.6 | — |
| LALA | 71.2 | +0.6 |
| LALA-DA | 68.7 | −1.9 |
| LALA-PG | 69.6 | −1.0 |

As can be seen from Table 3, the Tₘ value of the LALA variant was 0.6° C. higher than that of IgG1 WT, while the Tₘ values of the LALA-DA variant and the LALA-PG variant were 1.9° C. and 1.0° C. lower than that of IgG1 WT, respectively.

### Example 1: Evaluation of Binding Activity of Anti-EGFR Antibodies with L234A/L235A and D265 Mutations to Human FcγRI

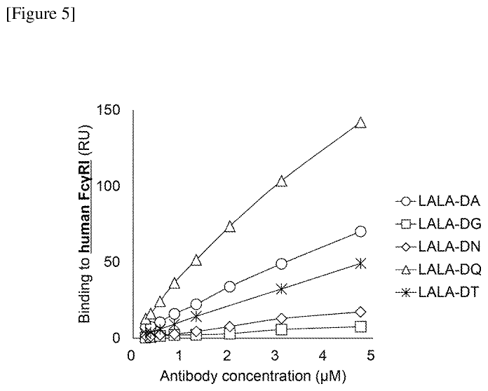

The binding activity of anti-EGFR antibody with L234A/L235A and D265 mutations to human FcγRI was evaluated in the same manner as in Comparative Example 1. The Fc region of the anti-EGFR antibody panitumumab, which is originally a human IgG2 antibody, was replaced with that of human IgG1 (IgG1 WT). The variants used for the evaluation were the LALA-DA variant of IgG1 WT; a variant obtained by introducing L234A/L235A/D265G (LALA-DG) mutations into the Fc region of IgG1 WT; a variant obtained by introducing L234A/L235A/D265N (LALA-DN) mutations into the Fc region of IgG1 WT; a variant obtained by introducing L234A/L235A/D265Q (LALA-DQ) mutations into the Fc region of IgG1 WT; and a variant obtained by introducing L234A/L235A/D265T (LALA-DT) mutations in the Fc region of IgG1 WT. The noted mutations were introduced into both CH2 domains of the Fc region. FIG. 5 shows a graph in which the RU values are plotted against each of the tested antibody concentrations (0.3, 0.4, 0.6, 0.9, 1.3, 2.0, 3.1, and 4.7 μM). Table 4 below shows the RU values obtained at an antibody concentration of 4.7 μM.

TABLE 4
Binding Activity of Variants and IgG1 WT
to Various Human Fcγ Receptors

| | | | hFcγRIIa | |
|---|---|---|---|---|
| | hFcγRI | hFcγRIIa | (H167) | hFcγRIIb/c |
| LALA-DA | 7.0 × 10 | 7.3 × 10 | 2.4 × 10 | 2.0 × 10 |
| LALA-DG | 7.6 | 5.5 × 10 | 8.8 | 1.5 × 10 |
| LALA-DN | 1.7 × 10 | 5.0 × 10 | 2.1 × 10 | 1.4 × 10 |
| LALA-DQ | 1.4 × 10² | 6.0 × 10 | 3.4 × 10 | 1.5 × 10 |
| LALA-DT | 4.9 × 10 | 5.7 × 10 | 3.7 × 10 | 1.5 × 10 |

| | | hFcγRIIIa | |
|---|---|---|---|
| | hFcγRIIIa | (V176F) | hFcγRIIIb |
| LALA-DA | 0.4 | −2.2 | 0.3 |
| LALA-DG | −2.7 | −4.4 | −0.3 |
| LALA-DN | −3.3 | −5.0 | −1.0 |
| LALA-DQ | −2.1 | −3.5 | −0.6 |
| LALA-DT | −1.4 | −3.6 | −0.2 |

As shown in Table 4, with regard to the binding activity to the human FcγRI, the RU values of the LALA-DG variant and the LALA-DN variant obtained at the antibody concentration of 4.7 μM were about 1/10 and about ¼ of that of the LALA-DA variant, respectively. The RU value of the LALA-DQ variant was about twice of that of the LALA-DA variant.

### Example 2: Evaluation of Binding Activity of Anti-EGFR Antibodies with L234A/L235A and D265 Mutations to Various Human FcγRs

The binding activity of the anti-EGFR antibodies described in Example 1 to human FcγRIIa, FcγRIIa (H167), FcγRIIb/c, FcγRIIIa, FcγRIIIa (V176F), and FcγRIIIb were evaluated in the same manner as in Comparative Example 2. Table 4 above shows the RU values obtained at an antibody concentration of 4.7 μM.

As can be seen from Table 4, the RU values for human FcγRs obtained at an antibody concentration of 4.7 μM were almost the same for each of the variants, with the exception of human FcγRIIa (H167). The RU value of the LALA-DG variant for human FcγRIIa (H167) was half or less than that of the other variants.

### Example 3: Evaluation of Binding Activity of Anti-EGFR Antibodies with L234A/L235A and D265 Mutations to Cynomolgus FcγRI

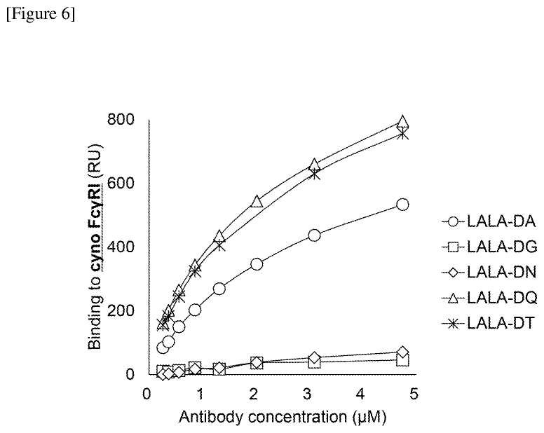

The binding activity of the anti-EGFR antibodies described in Example 1 to cynomolgus FcγRI were evaluated in the same manner as in Comparative Example 3. FIG. 6 shows a graph in which the RU values are plotted against each of the tested antibody concentrations (0.3, 0.4, 0.6, 0.9, 1.3, 2.0, 3.1, and 4.7 μM). Table 5 below shows the RU values for an antibody concentration of 4.7 μM.

TABLE 5
Binding Activity of Variants and IgG1 WT
to Various Cynomolgus Fcγ Receptors

| | CFcγRI | cFcγRIIa | cFcγRIIb | CFcγRIII |
|---|---|---|---|---|
| LALA-DA | 5.3 × 10² | 2.5 × 10 | 1.1 × 10² | 8.4 |
| LALA-DG | 4.6 × 10 | 1.9 × 10 | 8.9 × 10 | 3.8 |
| LALA-DN | 7.1 × 10 | 1.9 × 10 | 8.5 × 10 | 3.1 |
| LALA-DQ | 8.0 × 10² | 2.2 × 10 | 8.9 × 10 | 3.5 |
| LALA-DT | 7.6 × 10² | 2.4 × 10 | 9.3 × 10 | 4.8 |

As can be seen from Table 5, the RU value of the LALA-DG variant obtained at an antibody concentration of 4.7 μM was about 1/10 of that of the LALA-DA variant, and the RU value of the LALA-DN variant was about ¼ of that of the LALA-DA variant. On the other hand, the RU values of the LALA-DQ and LALA-DT variants were 1.4 times or more that of the LALA-DA variant.

### Example 4: Evaluation of Binding Activity of Anti-EGFR Antibodies with L234A/L235A and D265 Mutations to Various Cynomolgus FcγRs

The binding activity of the anti-EGFR antibodies described in Example 1 to cynomolgus FcγRIIa, FcγRIIb and FcγRIII were evaluated in the same manner as in Comparative Example 4. Table 5 above shows the RU values obtained at an antibody concentration of 4.7 μM.

As can be seen from Table 5, the RU values for cynomolgus FcγRIIa, FcγRIIb, and FcγRIII obtained at an antibody concentration of 4.7 μM were almost the same for each of the variants.

### Example 5: Evaluation of Thermal Stability of Anti-EGFR Antibodies with L234A/L235A and D265 Mutations

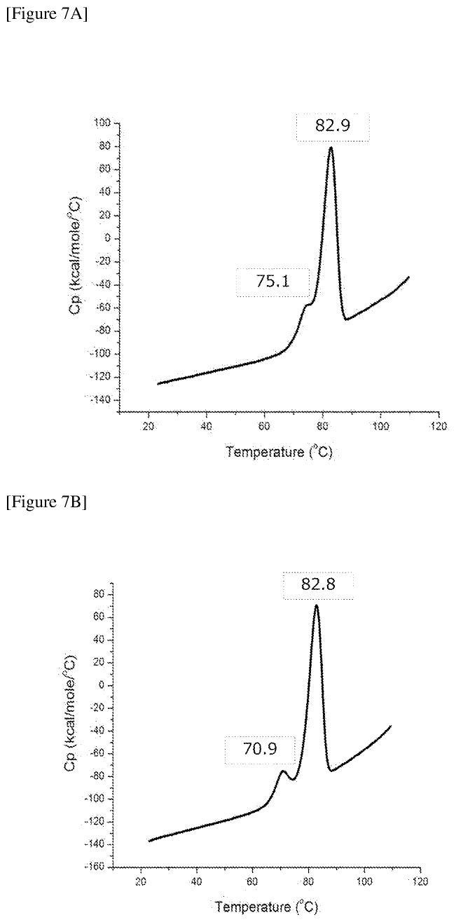

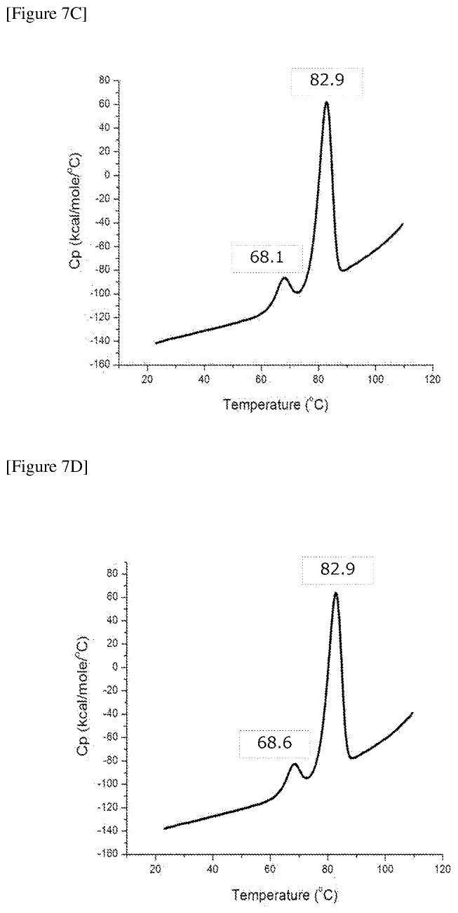

The thermal stability of each CH2 domain of IgG1 WT of the anti-EGFR antibody panitumumab and the variants described in Example 1 was evaluated in the same manner as in Comparative Example 5. FIGS. 7A to 7D show the changes in heat capacity and Tₘ values for the CH2 domain of the LALA-DG variant, LALA-DN variant, LALA-DQ variant, and LALA-DT variant of the anti-EGFR antibody. Table 6 below shows the Tₘ value for the CH2 domain for each of the test antibodies and the difference in Tₘ values (ΔTₘ) between each variant and IgG1 WT.

TABLE 6
Tₘ for the CH2 Domain of Variants and IgG1 WT and the
Difference in Tₘ (ΔTₘ) Between the Variant and IgG1 WT

| | CH2 Tₘ (° C.) | ΔTₘ (° C.) |
|---|---|---|
| IgG1 WT | 70.6 | — |
| LALA-DA | 68.7 | −1.9 |
| LALA-DG | 75.1 | +4.5 |
| LALA-DN | 70.9 | +0.3 |
| LALA-DQ | 68.1 | −2.5 |
| LALA-DT | 68.6 | −2.0 |

As shown in Table 6, the Tₘ values for the LALA-DQ variant and the LALA-DT variant, similar to the LALA-DA variant, were about 2° C. lower than that of IgG1 WT. The Tₘ value of the LALA-DG variant was 4.5° C. higher than that of IgG1 WT, and the Tₘ value of the LALA-DN variant was 0.3° C. higher than that of IgG1 WT.

### Example 6: Evaluation of Antigen-Binding Activity of a LALA-DG Variant of an Anti-EGFR Antibody

Biacore™ T200 was used to evaluate the EGFR-binding activity of IgG1 WT and LALA and LALA-DG variants of the anti-EGFR antibody panitumumab. The anti-EGFR antibody as a ligand was captured on a sensor chip on which an anti-human IgG Fc antibody was immobilized, and EGFR was flowed over as an analyte. The interaction between the ligand and the analyte was then measured by SPR method.

Briefly, an anti-human IgG Fc antibody contained in the Human Antibody Capture Kit (Cytiva, Marlborough, MA) was immobilized on a Sensor Chip CM5 according to the manufacturer's instructions, and then each of the anti-EGFR antibodies prepared at 2 μg/mL in HBS-EP+ was captured by contacting it with the sensor chip at a flow rate of 10 μL/min for 30 seconds. Subsequently, Human EGFR Protein (ACROBiosystems, Newark, DE) prepared at concentrations of 12, 37, 110, 330, 1000 μM in HBS-EP+ was added to the sensor chip at a flow rate of 30 μL/min for 120 seconds, and then dissociated for 1500 seconds. The dissociation constant K_{D} for each of the antibodies against EGFR was calculated from the resulting sensorgram by single cycle kinetics analysis. The results are shown in Table 7 below.

TABLE 7
Dissociation Constant K_{D} for Anti-EGFR
antibodies against EGFR antigen

| | K_{D} (pM) |
|---|---|
| IgG1 WT | 27 |
| LALA | 24 |
| LALA-DG | 26 |

As can be seen from Table 7, the K_{D} values for the antibodies against EGFR were 27, 24, and 26 nM for IgG1 WT, LALA variant, and LALA-DG variant, respectively, which were almost the same regardless of the presence or absence of the mutations and the type thereof.

### Example 7: Evaluation of Binding Activity of a LALA-DG Variant and a LALA-DGPA Variant of an Anti-EGFR Antibody to Neonatal Fc Receptor (FcRn)

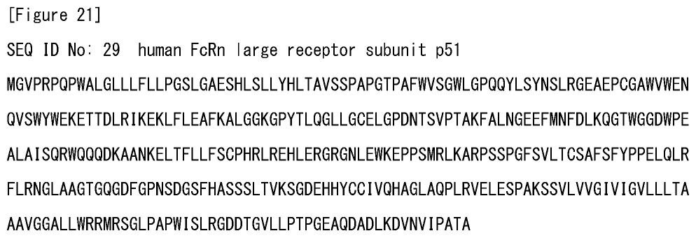

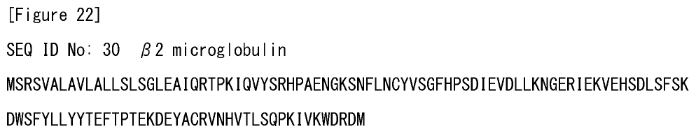

An Octet® RED384 system (Molecular Devices, LLC, San Jose, CA) was used to evaluate the binding activity of the anti-EGFR antibodies described in Example 6 to human FcRn (human neonatal receptor large subunit p51/Accession #: P55899/SEQ ID No: 29/FIG. 21) and beta2-microglobulin (Accession #: NP_004039.1/SEQ ID No: 30/FIG. 22). A Bio-Layer Interferometry method was used to measure the binding reaction between the antibody and FcRn upon contacting a Streptavidin Biosensor (Molecular Devices, LLC), on which biotinylated FcRn was captured, with a solution of the anti-EGFR antibody, and the dissociation reaction between the antibody and FcRn upon transferring the biosensor to another solution containing no antibody. Both the binding and dissociation reactions were carried out in a buffer solution at pH 6.0, and the dissociation constant K_{D} at pH 6.0 was calculated from the resulting sensorgram of the binding and dissociation reactions. In addition, the binding reaction was carried out in a buffer solution at pH 6.0, and the dissociation reaction was carried out in a buffer solution at pH 7.4. The dissociation rate constant k_{d} at pH 7.4 was calculated from the resulting sensorgram of the dissociation reaction. Table 8 below shows the calculated K_{D} and k_{d} values for the anti-EGFR antibodies. The concentration of the anti-EGFR antibody solution was set at three points, namely 1.7, 6.9 and 28 nM. A solution of 20 mM Bis-Tris, 150 mM NaCl, and 0.05% Surfactant P20 at pH 6.0 or 7.4 was used as the buffer solution.

TABLE 8
Dissociation Constant K_{D} and Dissociation Rate Constant
k_{d} for Anti-EGFR Antibodies for Human FcRn

| | K_{D} (nM) | k_{d} (1/s) |
|---|---|---|
| | at pH 6.0 | at pH 7.4 |
| IgG1 WT | 5.8 | 2.1 |
| LALA | 5.5 | 2.8 |
| LALA-DG | 5.8 | 2.2 |
| LALA-DGPA | 5.0 | 2.3 |

As can be seen from Table 8, the K_{D} value at pH 6.0 for all of IgG1 WT, LALA variant, LALA-DG variant, and LALA-DGPA variant was 5-6 nM. In addition, the k_{d} values at pH 7.4 were almost the same, i.e., 2.1 (1/s) for IgG1 WT, and 2.8, 2.2, 2.3 (1/s), respectively for the variants.

### Example 8: Pharmacokinetic Test of a LALA-DG Variant of an Anti-EGFR Antibody in Mouse

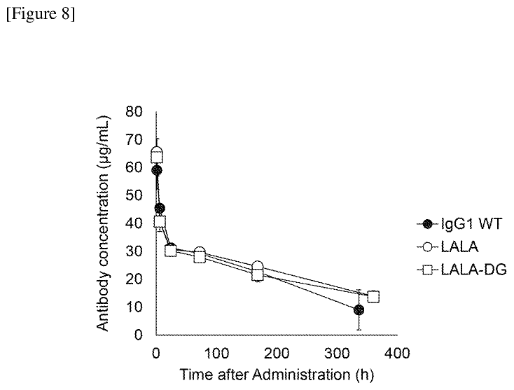

A pharmacokinetic test for the anti-EGFR antibodies described in Example 6 was conducted in mouse. Each of the anti-EGFR antibodies was intravenously administered to mice at 3 mg/kg, and blood was then collected from the tail vein after 1, 6, 24, 72, 168, 336 and 360 hours using a heparin-treated hematocrit tube under isoflurane anesthesia, followed by centrifugation at 4° C. and 15000 rpm for 5 minutes to provide plasma. The amount of the anti-EGFR antibody in the resulting plasma was measured using a fully automatic immunoassay platform Gyrolab™ xP Workstation (Gyros Protein Technologies AB, Uppsala, Sweden). A biotin-labeled anti-human IgG goat antibody (SouthernBiotech, Birmingham, AL) was used for capture of the anti-EGFR antibody, and an Alexa Fluor 647-labeled anti-human IgG goat antibody (SouthernBiotech) was used for detection. FIG. 8 shows a graph in which the blood concentrations of the anti-EGFR antibodies are plotted against time after administration.

As can be seen from FIG. 8, no significant difference was observed in the changes in the blood concentrations of IgG1 WT, LALA and LALA-DG variants of the anti-EGFR antibody in mouse over time.

### Example 9: Evaluation of Binding Activity of an Anti-EGFR Antibody with L234A/L235A/D265G/P329A Mutations to Human FcγRI

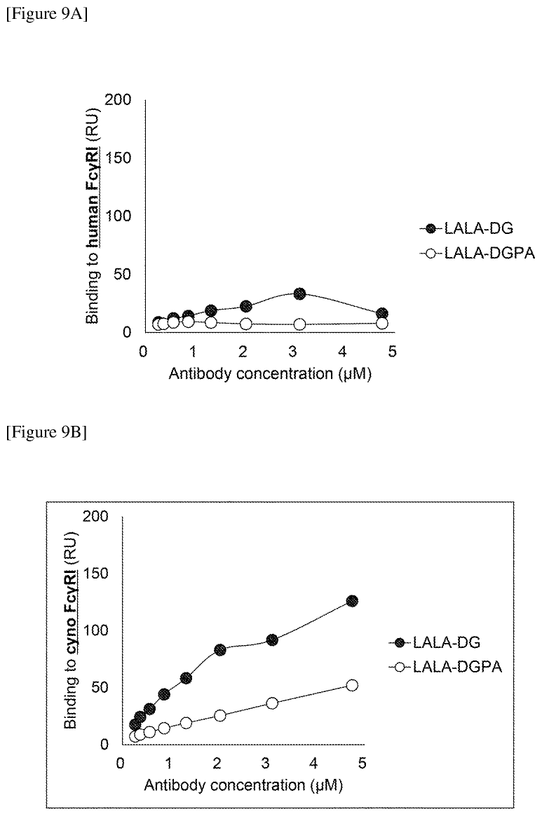

The binding activity of the LALA-DG variant of the anti-EGFR antibody panitumumab and a variant obtained by introducing L234A/L235A/D265G/P329A (LALA-DGPA) mutations into the Fc region of IgG1 WT, to human FcγRI was evaluated in the same manner as in Comparative Example 1. FIG. 9A shows a graph in which the RU values are plotted against each of the tested antibody concentrations (0.3, 0.4, 0.6, 0.9, 1.3, 2.0, 3.1, and 4.7 μM) for the LALA-DG variant (see Example 1) and the LALA-DGPA variant of the anti-EGFR antibody.

As can be seen from FIG. 9A, the LALA-DGPA variant showed a lower RU value for human FcγRI than the LALA-DG variant.

### Example 10: Evaluation of Binding Activity of an Anti-EGFR Antibody with L234A/L235A/D265G/P329A Mutations to Cynomolgus FcγRI

The binding activity of the anti-EGFR antibodies described in Example 9 to cynomolgus FcγRI were evaluated in the same manner as in Comparative Example 3. FIG. 9B shows a graph in which the RU values are plotted against each of the tested antibody concentrations (0.3, 0.4, 0.6, 0.9, 1.3, 2.0, 3.1, and 4.7 μM) for the LALA-DG variant (see Example 1) and the LALA-DGPA variant of the anti-EGFR antibody.

As can be seen from FIG. 9B, the RU value of the LALA-DGPA variant for cynomolgus FcγRI was lower than that of the LALA-DG variant.

### Example 11: Evaluation of Thermal Stability of an Anti-EGFR Antibody with L234A/L235A/D265G/P329A Mutations

The thermal stability of the anti-EGFR antibodies described in Example 9 was evaluated in the same manner as in Comparative Example 5. Table 9 below shows the Tₘ value for the CH2 domain of each of the LALA-DG variant and the LALA-DGPA variant of the anti-EGFR antibody. The Tm value of the CH2 domain of the LALA-DG variant is the same as in Table 6.

TABLE 9
Tₘ Value for the CH2 Domain of Anti-EGFR Antibodies

| | CH2 Tₘ (° C.) |
|---|---|
| LALA-DG | 75.3 |
| LALA-DGPA | 74.3 |

As shown in Table 9, the Tₘ value of the CH2 domain of the LALA-DGPA variant was 1° C. lower than that of the LALA-DG variant.

### Example 12: Evaluation of Binding Activity of a LALA-DG Variant of an Anti-CD70 Antibody to Various Human Fcγ Receptors

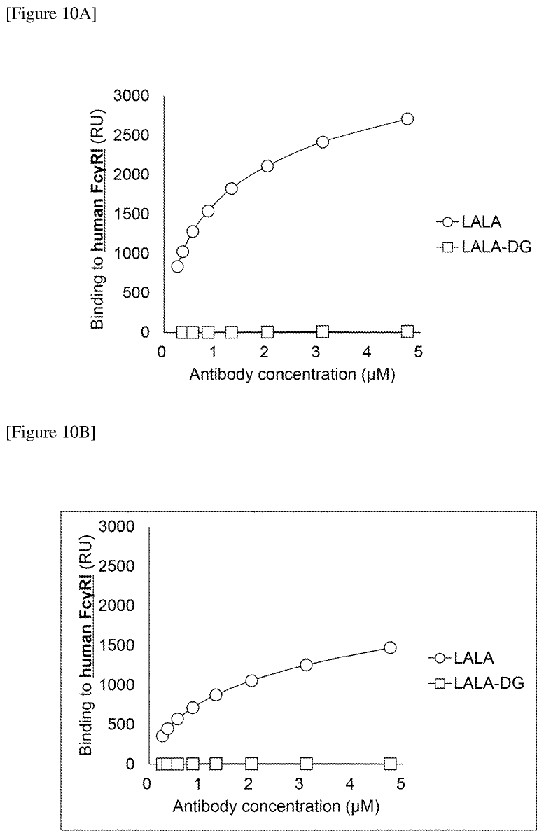

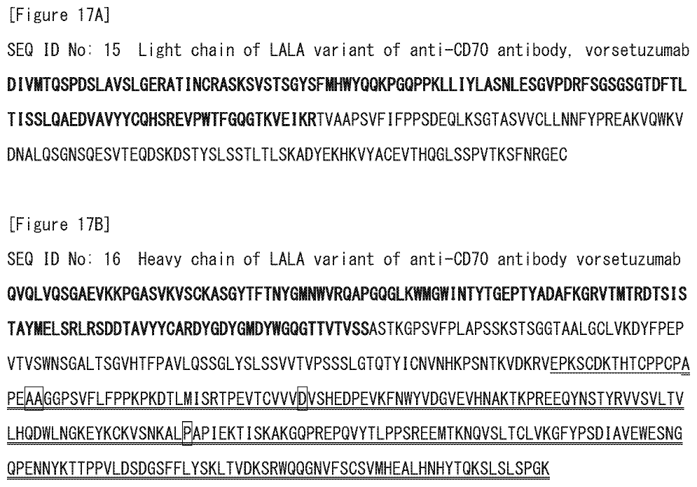

The binding activity of a LALA variant and a LALA-DG variant of an anti-CD70 antibody vorsetuzumab, which is an IgG1 antibody, to human FcγRI, were obtained and evaluated in the same manner as in Comparative Example 1. The amino acid sequences of the light chain and the heavy chain of the LALA variant of vorsetuzumab are represented by SEQ ID Nos: 15 and 16, respectively (see FIGS. 17A and 17B). FIG. 10A shows a graph in which the RU values are plotted against each of the tested antibody concentrations (0.3, 0.4, 0.6, 0.9, 1.3, 2.0, 3.1, and 4.7 μM). In addition, the method described in Comparative Example 2 was used to evaluate the binding activity of these antibodies to human FcγRIIa, FcγRIIb/c, FcγRIIIa, and FcγRIIIb. Table 10 shows the RU values obtained at an antibody concentration of 4.7 μM.

TABLE 10
Binding Activity of a LALA and a LALA-DG Variant of an Anti-CD70
Antibody and an Anti-LPS Antibody to Various Human Fcγ Receptors

| | | hFcγRI | hFcγRIIa | hFcγRIIb/c | hFcγRIIIa | hFcγRIIIb |
|---|---|---|---|---|---|---|
| Anti-CD70 | LALA | 2.7 × 10³ | 6.4 × 10 | 1.8 × 10 | 1.0 × 10² | 4.5 |
| Antibody | LALA-DG | 1.4 × 10 | 8.3 | 1.3 | −2.1 | −3.7 |
| Anti-LPS | LALA | 1.5 × 10³ | 3.4 × 10 | 7.8 | 7.6 × 10 | −1.4 |
| Antibody | LALA-DG | 4.8 | −2.2 | −2.6 | −7.2 | −7.1 |

The RU value for the LALA-DG variant for human FcγRI obtained at an antibody concentration of 4.7 μM was about 1/200 of that of the LALA variant. In addition, the RU value of the LALA-DG variant for the other human FcγRs obtained at an antibody concentration of 4.7 μM was lower than that of the LALA variant.

### Example 13: Evaluation of Binding Activity of a LALA-DG Variant of an Anti-LPS Antibody to Human Fcγ Receptors

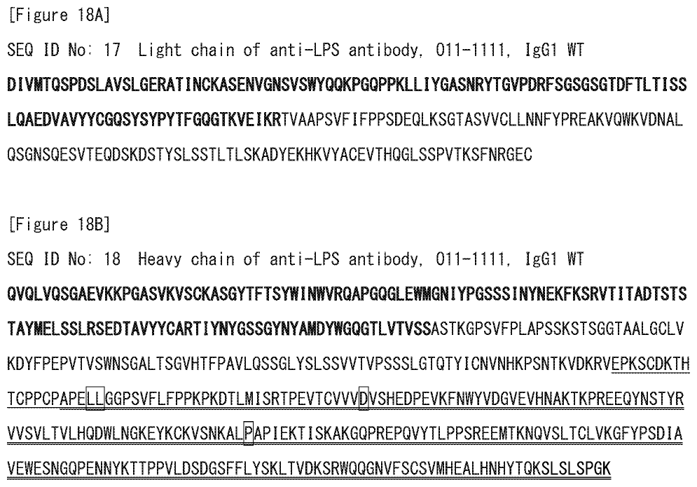

The binding activity of a LALA variant and a LALA-DG variant of an anti-LPS antibody O11-1111, which is an IgG1 antibody, to human FcγRI were obtained and evaluated in the same manner as in Comparative Example 1. The amino acid sequences of the light chain and the heavy chain of the 011-1111 antibody are represented by SEQ ID Nos: 17 and 18, respectively (see FIGS. 18A and 18B). FIG. 10B shows a graph in which the RU values are plotted against each of the tested antibody concentrations (0.3, 0.4, 0.6, 0.9, 1.3, 2.0, 3.1, and 4.7 μM). In addition, the method described in Comparative Example 2 was used to evaluate the binding activity of these antibodies to human FcγRIIa, FcγRIIb/c, FcγRIIIa, and FcγRIIIb. Table 10 above shows the RU values for an antibody concentration of 4.7 μM.

As can be seen from Table 10, the RU value of the LALA-DG variant for human FcγRI obtained at an antibody concentration of 4.7 μM was about 1/300 of that of the LALA variant. In addition, the RU value of the LALA-DG variant for the other human FcγRs obtained at an antibody concentration of 4.7 μM was lower than that of the LALA variant.

### Example 14: Evaluation of Thermal Stability of Various Antibodies with LALA-DG Mutations

The thermal stability of the following antibodies was evaluated in the same manner as Comparative Example 5. The antibodies evaluated were those the parent antibody (IgG1 WT) of the anti-LPS antibody, O11-1111, and the parent antibody (IgG1 WT) of the anti-CD70 antibody, the LALA and LALA-DG variants of each of these antibodies, and additionally antibodies derived from said variants each of which lacks any Fab regions. The LALA variant lacking Fab and the LALA-DG variant lacking Fab consist of the 222nd to 448th amino acids of SEQ ID No: 16; and the 104th to 330th amino acids of SEQ ID No. 5, respectively (see FIGS. 17B and 12B). Table 11 shows the Tₘ value for the CH2 domain of each of the tested antibodies.

TABLE 11
Tₘ Values for the CH2 Domains of Various Antibodies

| | CH2 Tₘ (° C.) | | |
|---|---|---|---|
| | Anti-CD70 Ab | Anti-LPS Ab | Fc (no Fabs) |
| IgG1 WT | — | 70.8 | — |
| LALA | 72.4 | 71.2 | 70.1 |
| LALA-DG | 76.3 | 74.5 | 73.7 |

As shown in Table 11, with regard to the anti-CD70 antibody, the Tₘ value for the CH2 domain of the LALA-DG variant was 3.9° C. higher than that of the LALA variant. With regard to the anti-LPS antibody, the Tₘ value for the CH2 domain of the LALA-DG variant was 3.7° C. higher than that of IgG1 WT and 3.3° C. higher than that of the LALA variant. With regard to the Fc region lacking Fabs, the Tₘ value for the CH2 domain of the LALA-DG variant was 3.6° C. higher than that of the LALA variant.

What is claimed is:

1. A polypeptide comprising one or more variant Fc regions, each of the variant Fc regions comprising two CH2 domains, wherein at least one CH2 domain in at least one of the variant Fc regions is a variant CH2 domain comprising amino acid substitutions at positions 234, 235 and 265 (all positions expressed by EU numbering), wherein the amino acid residues at positions 234, 235, and 265 of the variant CH2 domain are Ala, Ala, and Gly, respectively, or Ala, Ala, and Asn, respectively, and wherein each of the one or more variant Fc regions is a variant IgG1 Fc region, a variant IgG4 Fc region or a variant IgG1/IgG4 Fc region.

2. The polypeptide according to claim 1, further comprising an amino acid substitution at position 329 of the variant CH2 domain, wherein the amino acid residue at position 329 of the variant CH2 domain is Ala.

3. The polypeptide according to claim 1 or 2, wherein the polypeptide has reduced binding affinity to at least one Fcγ receptor (FcγR) as compared to a parent polypeptide comprising a parent CH2 domain without amino acid substitutions at positions 234, 235 and 265.

4. The polypeptide according to claim 3, wherein the polypeptide has reduced binding to one or more of an FcγRI, an FcγRII or an FcγRIII.

5. The polypeptide according to claim 3, wherein the polypeptide has reduced binding to an FcγRI, an FcγRII and an FcγRIII.

6. The polypeptide according to claim 4 or 5, wherein the FcγRI, FcγRII and FcγRIII are human FcγRI, FcγRII and FcγRIII.

7. The polypeptide according to claim 4 or 5, wherein the FcγRI, FcγRII and FcγRIII are cynomolgus FcγRI, FcγRII and FcγRIII.

8. The polypeptide according to claim 1 or 2, wherein the polypeptide has reduced binding affinity to an Fcγ receptor (FcγR) by 50% or more as compared to a parent polypeptide comprising a parent CH2 domain without amino acid substitutions at positions 234, 235, and 265.

9. The polypeptide according to claim 8, wherein the FcγR is a human FcγR or a cynomolgus FcγR.

10. The polypeptide according to claim 8, wherein the FcγR is a human FcγR.

11. The polypeptide according to any one of claims 8 to 10, wherein the FcγR is FcγRI.

12. The polypeptide according to any one of claims 1 to 11, wherein the polypeptide comprises one or more variant IgG1 Fc regions.

13. The polypeptide according to any one of claims 1 to 11, wherein the polypeptide comprises one variant Fc region.

14. The polypeptide according to any one of claims 1 to 11, wherein the polypeptide comprises one variant IgG1 Fc region.

15. The polypeptide according to any one of claims 1 to 14, wherein the amino acid residues at positions 234, 235, and 265 of the variant CH2 domain are Ala, Ala, and Gly, respectively.

16. The polypeptide according to any one of claims 1 to 14, wherein the amino acid residues at positions 234, 235, and 265 of the variant CH2 domain are Ala, Ala, and Asn, respectively.

17. The polypeptide according to claim 15 or 16, wherein a thermal denaturation midpoint temperature (Tm) of the variant CH2 domain is within 1° C. of the Tm of a parent CH2 domain without amino acid substitutions at positions 234, 235 and 265.

18. The polypeptide according to claim 15, wherein a thermal denaturation midpoint temperature (Tm) of the variant CH2 domain is higher by 1° C. or more than the Tm of a parent CH2 domain without amino acid substitutions at positions 234, 235 and 265.

19. The polypeptide according to claim 18, wherein the Tm of the variant CH2 domain is higher by 3° C. or more than the Tm of the parent CH2 domain.

20. The polypeptide according to any one of claims 1 to 19, wherein the polypeptide is an antibody or a functional fragment thereof.

21. The polypeptide according to claim 20, wherein the antibody or functional fragment thereof binds a tumor antigen.

22. A molecule comprising the polypeptide according to any one of claims 1 to 21.

23. The molecule according to claim 22, which is a fusion protein of the polypeptide according to any one of claims 1 to 21 and a heterologous polypeptide.

24. A polypeptide-drug conjugate comprising the polypeptide according to any one of claims 1 to 21 conjugated to one or more drugs or modifying agents.

25. The polypeptide-drug conjugate according to claim 24, wherein the drug is a chemotherapeutic agent or a cytotoxic agent.

26. The polypeptide-drug conjugate according to claim 24, wherein the modifying agent is a non-proteinaceous polymer.

27. A nucleic acid comprising a nucleotide sequence encoding the polypeptide according to any one of claims 1 to 21 or a portion thereof.

28. A host cell comprising the nucleic acid according to claim 27.

29. A method for producing the polypeptide according to any one of claims 1 to 21 comprising culturing the host cell according to claim 28 in a medium under conditions suitable for expression of the polypeptide, and recovering the polypeptide from the medium or the host cell.

30. A pharmaceutical composition comprising the polypeptide according to any one of claims 1 to 21, the molecule according to claim 22 or 23, or the polypeptide-drug conjugate according to any one of claims 24 to 26, and a pharmaceutically acceptable carrier.

31. A polypeptide according to any one of claims 1 to 21, a molecule according to claim 22 or 23, or a polypeptide-drug conjugate according to any one of claims 24 to 26, for use in therapy.

32. The polypeptide, molecule or polypeptide-drug conjugate for use according to claim 31, wherein the therapy comprises treatment of a cancer, an immune disease, an inflammatory disease or an infectious disease.


SEQUENCE LISTING (31 sequences)

SEQ ID NO: 1 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVGVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVGVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVNVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVNVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVGVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVGVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEAAGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEAAGGPSVFLFPPKPKDTLMISRTPEVTCVVVNVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEAAGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLASSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 218 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 448 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, synthetic construct
DIVMTQSPDSLAVSLGERATINCKASENVGNSVSWYQQKPGQPPKLLIYGASNRYTGVPDRFSGSGSGTDFTLTISSLQAEDVAVYYCGQSYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 455 aa, synthetic construct
QVQLVQSGAEVKKPGASVKVSCKASGYTFTSYWINWVRQAPGQGLEWMGNIYPGSSSINYNEKFKSRVTITADTSTSTAYMELSSLRSEDTAVYYCARTIYNYGSSGYNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 20 - protein, 317 aa, Homo sapiens [sequence omitted]

SEQ ID NO: 21 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 22 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 23 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 24 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 25 - protein, 374 aa, Macaca fascicularis
MWFLTALLLWVPVDGQVDTTKAVITLQPPWVSVFQEETVTLQCEVPRLPGSSSTQWFLNGTATQTSTPSYRITSASVKDSGEYRCQRGPSGRSDPIQLEIHRDWLLLQVSSRVFTEGEPLALRCHAWKDKLVYNVLYYQNGKAFKFFYRNSQLTILKTNISHNGAYHCSGMGKHRYTSAGVSVTVKELFPAPVLNASVTSPLLEGNLVTLSCETKLLLQRPGLQLYFSFYMGSKTLRGRNTSSEYQILTARREDSGFYWCEATTEDGNVLKRSPELELQVLGLQLPTPVWLHVLFYLVVGIMVLVNTGLWVTIRKELKRKKKWNLEISLDSAHEKKVTSSLQEDRHLEEELKSQEQEKEQLQEGVHRKEPEEAK

SEQ ID NO: 26 - protein, 310 aa, Macaca fascicularis
MSQNVCPGNLWLLQPLTVLLLLASADSQTAPPKAVLKLEPPWINVLREDSVTLTCGGAHSPDSDSTQWFHNGNRIPTHTQPSYRFKANNNDSGEYRCQTGRTSLSDPVHLTVLSEWLALQTPHLEFREGETIMLRCHSWKDKPLIKVTFFQNGIAKKFSHMDPNFSIPQANHSHSGDYHCTGNIGYTPYSSKPVTITVQVPSVGSSSPMGIIVAVVTGIAVAAIVAAVVALIYCRKKRISANSTDPVKAARFEPLGRQTIALRKRQLEETNNDYETADGGYMTLNPRAPTDDDRNIYLTLSPNDYDNSNN

SEQ ID NO: 27 - protein, 294 aa, Macaca fascicularis
MGILSFLPVLATESDWADCKSSQPWGHMLLWTAVLFLAPVAGTPAAPPKAVLKLEPPWINVLREDSVTLTCGGAHSPDSDSTQWFHNGNLIPTHTQPSYRFKANNNDSGEYRCQTGRTSLSDPVHLTVLSEWLALQTPHLEFREGETILLRCHSWKDKPLIKVTFFQNGISKKFSHMNPNFSIPQANHSHSGDYHCTGNIGYTPYSSKPVTITVQVPSMGSSSPIGIIVAVVTGIAVAAIVAAVVALIYCRKKRISANPTNPDEADKVGAENTITYSLLMHPDALEEPDDQNRV

SEQ ID NO: 28 - protein, 254 aa, Macaca fascicularis
MWQLLLPTALLLLVSAGMRAEDLPRAVVFLEPQWYRVLEKDRVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQVSFCLVMVLLFAVDTGLYFSMKKSIPSSTRDWEDHKFKWSKDPQDK

SEQ ID NO: 29 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 30 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 31 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK